(19) United States
(12) Patent Application Publication
(10) Pub. No.: US 2015/0299652 A1
(43) Pub. Date: Oct. 22, 2015

(54) COMPOSITIONS AND METHODS FOR GENERATING NEURAL CREST STEM CELLS AND SENSORY NEURONS

(71) Applicant: UNIVERSITY OF CENTRAL FLORIDA RESEARCH FOUNDATION, INC., Orlando, FL (US)

(72) Inventors: Xiufang GUO, Orlando, FL (US); Severo SPRADLING, Orlando, FL (US); James J. HICKMAN, Orlando, FL (US)

(73) Assignee: UNIVERSITY OF CENTRAL FLORIDA RESEARCH FOUNDATION INC., Orlando, FL (US)

(21) Appl. No.: 14/648,190

(22) PCT Filed: Nov. 27, 2013
(86) PCT No.: PCT/US2013/072382

§ 371 Date: May 28, 2015

Related U.S. Application Data
(60) Provisional application No. 61/732,042, filed on Nov. 30, 2012.
(60) Provisional application No. 61/732,574, filed on Dec. 3, 2012.
(60) Provisional application No. 61/784,923, filed on Mar. 14, 2013.

(51) Int. Cl. C12N 5/0793; C12N 5/079; C12N 5/0797

(52) U.S. Cl. CPC C12N 5/062; C12N 5/0623; C12N 5/0622; C12N 2506/08; C12N 2533/52; C12N 2533/50; C12N 2501/115; C12N 2501/11; C12N 2533/30; C12N 2501/415; C12N 2501/13; C12N 2501/999

(57) ABSTRACT

In an aspect, the invention relates to compositions, methods, and kits for generating neural crest stem cells, sensory neurons, and Schwann cells. This abstract is intended as a scanning tool for purposes of searching in the particular art and is not intended to be limiting of the present invention.

### CROSS-REFERENCE TO RELATED APPLICATIONS

This application claims priority to U.S. Provisional Patent Application No. 61/732,042 filed Nov. 30, 2012, to U.S. Provisional Patent Application No. 61/732,574 filed Dec. 3, 2012, and to U.S. Provisional Patent Application No. 61/784,923 filed Mar. 14, 2013, each of which is incorporated herein by reference in its entirety

### STATEMENT REGARDING FEDERALLY SPONSORED RESEARCH OR DEVELOPMENT

This invention was made with government support under R01-NS050452 and R01-EB009429 awarded by the National Institutes of Health. The government has certain rights in the invention.

### BACKGROUND

Sensory neurons are responsible for conveying internal, external, and environmental stimuli to the central nervous system (CNS). Sensory neurons act as signal initiators in all reflex responses, and constitute an indispensable component for the correct function of the nervous system. Sensory neurons can be damaged or injured through a variety of means such as traumatic injury (Fernandez-Valle et al., 1995), infection, toxin exposure, metabolic disease, immune system disorders, cancer and chemotherapy (Sheikh et al., 2010), and heredity (Groves et al., 2005; Sghirlanzoni et al., 2005). The subsequent cellular dysfunction caused by such damage or injury is associated with symptoms ranging from abnormal sensation to numbness and pain to loss of coordination in voluntary movement (Sheikh et al., 2010; Groves et al., 2005; Sghirlanzoni et al., 2005).

In general, culturing embryonic stem cells (ESCs) is a lengthy process that requires the handling of aggregated embryoid bodies and neurospheres. To date, such ESC approaches have only produced small yields of the differentiated cellular phenotypes. In such cultures, neural crest cells are typically found interspersed with neural rosettes, and therefore, cell-sorting is required to obtain a highly enriched cell population (Lee et al., 2007; Lee et al., 2010; Brokham et al., 2008). The generation of sensory neurons from ESCs requires a series of stages: (1) the induction of neural ectoderm, which distinguishes the developing nervous system from other systems; (2) the induction of neural crest cell fate, which segregates the peripheral nervous system (PNS) from the CNS, and (3) the differentiation of sensory neurons, which distinguishes them from other neural crest derivatives. Currently available culturing protocols lack the ability to generate a population of functional sensory neurons without employing a series of technically challenging, time-consuming, and contaminating steps.

Therefore, there is still a scarcity of compositions, methods, and kits that effectively provide an in vitro source of human (and non-human) sensory neurons, which neurons would generate invaluable material for use in functional human (and non-human) disease models, in pathological studies and drug screening, and in regenerative medicine. These needs and other needs are satisfied by the compositions, methods, and kits disclosed herein.

### SUMMARY

Disclosed herein are method of generating sensory cells or neural crest stem cells, comprising proliferating a population of neural progenitor cells; dissociating the population of proliferated neural progenitor cells; replating the population of dissociated neural progenitor cells; expanding the population of replated neural progenitor cells; and initiating differentiation of the population of expanded neural progenitor cells into sensory neurons or neural crest stem cells

Disclosed herein are methods of generating sensory neurons, comprising proliferating a population of neural progenitor cells; dissociating and replating the population of neural progenitor cells; expanding the population of neural progenitor cells; and initiating differentiation of the population of neural progenitor cells, whereby the population of neural progenitor cells differentiates into sensory neurons.

Disclosed herein are methods of generating sensory neurons, comprising proliferating a population of neural progenitor cells; dissociating the population of proliferated neural progenitor cells; replating the population of dissociated neural progenitor cells; expanding the population of replated neural progenitor cells; initiating differentiation of the population of expanded neural progenitor cells; and promoting differentiation of the population of differentiating neural progenitor cells, whereby the population of neural progenitor cells differentiates into sensory neurons.

Disclosed herein are methods of generating sensory neurons, comprising proliferating a population of neural crest stem cells; and initiating differentiation of the population of neural crest stem cells into sensory neurons.

Disclosed herein are sensory neurons made by a disclosed method of generating sensory neurons, wherein the method comprises a population of neural progenitor cells.

Disclosed herein are uses of sensory neurons made by a disclosed method of generating sensory neurons, wherein the method comprises a population of neural progenitor cells.

Disclosed herein are methods of generating neural crest stem cells, comprising proliferating a population of neural progenitor cells; dissociating and replating the population of neural progenitor cells; expanding the population of neural progenitor cells; and initiating differentiation of the population of neural progenitor cells, whereby the population of neural progenitor cells differentiates into neural crest stem cells.

Disclosed herein are methods of generating neural crest stem cells, comprising proliferating a population of neural progenitor cells; dissociating the population of proliferated neural progenitor cells; replating the population of dissociated neural progenitor cells; expanding the population of replated neural progenitor cells; initiating differentiation of the population of expanded neural progenitor cells, dissociating the population of differentiating neural progenitor cells; and replating the population of dissociated and differentiating neural progenitor cells, whereby the population of neural progenitor cells differentiates into neural crest stem cells.

Disclosed herein are neural crest stem cells made by a disclosed method of generating neural crest stem cells.

Disclosed herein are uses of neural crest stem cells made by a disclosed method of generating neural crest stem cells.

Disclosed herein are methods of generating sensory neurons, comprising proliferating a population of neural crest stem cells; and initiating differentiation of the population of neural crest stem cells, whereby the population of neural crest stem cells differentiates into sensory neurons.

Disclosed herein are sensory neurons made by a disclosed method of generating sensory neurons, wherein the method comprises a population of neural crest stem cells.

Disclosed herein are uses of sensory neurons made by a disclosed method of generating sensory neurons, wherein the method comprises a population of neural crest stem cells.

Disclosed herein are methods of generating Schwann cells, the methods comprising proliferating a population of neural crest stem cells; and initiating the differentiation of the population of neural crest stem cells, whereby the population of neural crest stem cells differentiates into Schwann cells.

Disclosed herein are methods of generating Schwann cells, comprising proliferating a population of neural crest stem cells; and initiating the differentiation of the population of neural crest stem cells into Schwann cells.

Disclosed herein are Schwann cells made generated by a disclosed method for generating Schwann cells.

Disclosed herein are uses of Schwann cell generated by a disclosed method for generating Schwann cells.

Disclosed herein are kits comprising a population of neural progenitor cells; and instructions for differentiating the population of neural progenitor cells into sensory neurons.

Disclosed herein are kits comprising a population of neural progenitor cells; and instructions for differentiating the population of neural progenitor cells into neural crest stem cells.

Disclosed herein are kits comprising a population of neural crest stem cells; and instructions for differentiating the population of neural crest stem cells into sensory neurons, Schwann cells, or both.

### BRIEF DESCRIPTION OF THE FIGURES

The accompanying Figures, which are incorporated in and constitute a part of this specification, illustrate several aspects of the invention and together with the description serve to explain the principles of the invention.

FIG. 1 shows immunocytochemical characterization of the human neural progenitor cell line hNP1. (A) shows that hNP1 cells were immunostained with the neural progenitor markers SOX1 and nestin. All cells were positive for both markers. hNP1 cells were immunostained with the neural crest cell marker HNK1 in (B), the neuronal marker βIII Tubulin in (C), and the peripheral neuronal marker peripherin in (D). While a small number of cells were positive for HNK1, all of the cells were negative for βIII tubulin and peripherin.

FIG. 2 shows phase contrast images of the cultures before and after the sensory neuron induction. (A) show the hNP1 culture before sensory induction. (B) shows hNP1 culture 10 days after sensory induction. (C) shows the hNP1 culture 30 days after sensory induction. As shown in (A)-(C), neuronal clusters and axonal bundles, which resemble rat DRG cell cultures, were observed. For comparison, (D) shows an image of a rat embryonic DRG cell culture at 7 DIV.

FIG. 3 shows an abundant number of sensory neurons after differentiation. As shown in (A), day 14 differentiated cultures were co-immunostained with the sensory neuronal marker Brn3a and the neuronal marker βIII tubulin. As shown in (B), day 14 differentiated cultures were co-immunostained with the sensory neuronal marker Brn3a and the peripheral neuronal marker peripherin. (C) shows that on day 14, the culture contained sensory neurons at different developmental stages as demonstrated with immunostaining for peripherin: (i): bipolar, (ii) mid-stage, and (iii) pseudo-unipolar.

FIG. 4 shows generation of Schwann cells from the differentiated culture. Immunostaining of a day 38 culture with the Schwann cell marker S100 demonstrated a significant number of Schwann cells in the culture. Schwann cells were located either within the neuronal clusters (A) or Schwann cells were located along the axonal bundles (B). The neuronal clusters and axonal bundles were marked by peripherin immunostaining.

FIG. 5 shows the identities of the derived peripheral neurons as analyzed with immunocytochemistry. (A) shows that in a 38 DIV culture, no cell was positive for MASH1 (the autonomic neuronal marker). (B) shows that in a 50 DIV culture, most of the peripheral neurons were positive for parvalbumin (a marker for type I sensory neurons). (C) shows that in a 25 DIV culture, vGluT1 signal was observed abundantly in derived neurons. (D) shows that in a 78 DIV culture, little substance P staining was observed.

FIG. 6 shows the electrophysiological recordings from differentiated sensory neurons. Using sensory neurons in the differentiated culture, (A) shows a representative voltage clamp, (B) shows a representative current clamp, and (C) shows a representative action potential trace. (D) shows an image of the recorded cell.

FIG. 7 shows an analysis of neural crest stem cells isolated from culture. (A) shows immunostaining of neural crest cells (passage 4) with the neural crest markers HNK1 and P75, which demonstrated an enriched neural crest cell population. (B) shows phase contrast images of neural crest cells (a) before induction, (b) after sensory neuronal induction, and (c) after Schwann cell induction. (C) shows peripheral neurons derived from neural crest cells (P4) after 15 days of sensory neuron induction as confirmed by immunostaining for peripherin. (D) shows Schwann cells derived from neural crest cells (P4) after 15 days as confirmed by immunostaining for S100 and GFAP.

### DESCRIPTION

The present invention can be understood more readily by reference to the following detailed description of the invention and the Examples included therein.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### A. DEFINITIONS

As used in the specification and the appended claims, the singular forms “a,” “an” and “the” include plural referents unless the context clearly dictates otherwise.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” can mean that the subsequently described event or circumstance can or cannot occur, and that the description includes instances where said event or circumstance occurs and instances where it does not.

As used herein, the term “subject” can refer to the target of administration, e.g., an animal. In an aspect, a subject can be a human or a non-human animal. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In an aspect, the subject is a human being. In an aspect, the subject is a non-human subject

As used herein, the term “subject” can also refer to the source of a material, such as a biological material, for example, cells, tissues, organs, etc. In an aspect, a subject can be a human or a non-human animal. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In an aspect, the subject that is a source of a material is a human being or a non-human being.

In an aspect, a subject can be afflicted with one or more diseases or disorders, such as, for example, a CNS (central nervous system) or PNS (peripheral nervous system) disease or disorder. The terms “CNS disease” or “CNS disorder” refer to neurological and/or psychiatric changes in the CNS, e.g., brain and spinal cord, which changes manifest in a variety of symptoms. Examples of CNS diseases or disorders include, but are not limited to, the following: migraine headache; cerebrovascular deficiency; psychoses including paranoia, schizophrenia, attention deficiency, and autism; obsessive/compulsive disorders including anorexia and bulimia; convulsive disorders including epilepsy and withdrawal from addictive substances; cognitive diseases including Parkinson's disease and dementia; and anxiety/depression disorders such as anticipatory anxiety (e.g., prior to surgery, dental work and the like), depression, mania, seasonal affective disorder (SAD); and convulsions and anxiety caused by withdrawal from addictive substances such as opiates, benzodiazepines, nicotine, alcohol, cocaine, and other substances of abuse. Further examples of CNS diseases and disorders include, but are not limited to, the following: Abercrombie's degeneration, Acquired epileptiform aphasia (Landau-Kleffner Syndrome), Acute Disseminated Encephalomyelitis, Adrenoleukodystrophy, Agnosia, Alexander Disease, Alpers' Disease, Alternating Hemiplegia, Amyotrophic Lateral Sclerosis, Angelman Syndrome, Ataxia Telangiectasia, Ataxias and Cerebellar/Spinocerebellar Degeneration, Attention Deficit Disorder, Binswanger's Disease, Canavan Disease, Cerebral Hypoxia, Cerebro-Oculo-Facio-Skeletal Syndrome, Charcot-Marie-Tooth, Chronic Inflammatory Demyelinating Polyneuropathy (CIDP), Corticobasal Degeneration, Creutzfeldt-Jakob disease, Degenerative knee arthritis, Diabetic neuropathy, Early Infantile Epileptic Encephalopathy (Ohtahara Syndrome), Epilepsy, Friedreich's Ataxia, Guillain-Barre Syndrome (GBS), Hallervorden-Spatz Disease, Huntington's Disease, Krabbe Disease, Kugelberg-Welander Disease (Spinal Muscular Atrophy), Leigh's Disease, Lennox-Gastaut Syndrome, Machado-Joseph Disease, Macular degeneration, Monomelic Amyotrophy, Multiple Sclerosis, Neuroacanthocytosis, Niemann-Pick disease, Olivopontocerebellar Atrophy, Paraneoplastic Syndromes, Parkinson's Disease, Pelizaeus-Merzbacher Disease, Pick's Disease, Primary Lateral Sclerosis, Progressive Locomotor Ataxia (Syphilitic Spinal Sclerosis, Tabes Dorsalis), Progressive Supranuclear Palsy, Rasmussen's Encephalitis, Rett Syndrome, Tourette's Syndrome, Usher syndrome, West syndrome (Infantile Spasms), and Wilson Disease. General characteristics of such diseases are known in the art. The skilled person can identify additional CNS diseases and disorders known in the art without undue experimentation.

As used herein, the terms “PNS disease” or “PNS disorder” can refer to a disease, illness, condition, or disorder that affects part or all of the peripheral nervous system. The PNS can comprise all the nerves in your body, aside from the ones in the brain and spinal cord. The PNS can act as a communication relay between the brain and the extremities. Unlike the CNS, the PNS is not protected by bone or the blood-brain barrier, which renders it exposed to toxins and mechanical injuries. Generally, the PNS can be divided into the somatic nervous system and the autonomic nervous system. As known to the art, there are over 100 types of PNS diseases and disorders. The causes of these PNS diseases or disorders include, but are not limited to, the following: diabetes, genetic predispositions (hereditary causes); exposure to toxic chemicals, alcoholism, malnutrition, inflammation (infectious or autoimmune), injury, and nerve compression; and by taking certain medications such as those used to treat cancer and HIV/AIDS. PNS diseases and disorders include anesthesia, hyperesthesia, paresthesia, and neuralgia. PNS diseases and disorders include, but are not limited to, the following: accessory nerve disorder, acrodynia, hand-arm vibration syndrome, amyloid neuropathies, anesthesia dolorosa, anti-mag peripheral neuropathy, autonomic dysreflexia, axillary nerve dysfunction, axillary nerve palsy, brachial plexus neuropathies, carpal tunnel syndrome, Charcot-Marie-Tooth disease, chronic solvent-induced encephalopathy, CMV polyradiculomyelopathy, complex regional pain syndromes, congenital insensitivity to pain with anhidrosis, diabetic neuropathies, dysautonomia, facial nerve paralysis, facial palsy, familial dysautonomia, Guillain-Barre syndrome, hereditary sensory and autonomic neuropathy, Homer's syndrome, Isaacs syndrome, ischiadica, leprosy, mononeuropathies, multiple system atrophy, myasthenia gravis, myotonic dystrophy, nerve compression syndrome, nerve injury, neuralgia, neuritis, neurofibromatosis, orthostatic hypotension, orthostatic intolerance, primary autonomic failure, pain insensitivity (congenital), peripheral nervous system neoplasms, peripheral neuritis, peripheral neuropathy, piriformis syndrome, plexopathy, polyneuropathies, polyneuropathy, post-herpetic neuralgia, postural orthostatic tachycardia syndrome, pronator teres syndrome, proximal diabetic neuropathy, pudendal nerve entrapment, pure autonomic failure, radial neuropathy, radiculopathy, sciatica, Tarlov cysts, thoracic outlet syndrome, trigeminal neuralgia, ulnar neuropathy, vegetative-vascular dystonia, Villaret's syndrome, Wartenberg's syndrome, and winged scapula.

As used herein, the term “diagnosed” can mean having been subjected to a physical, psychological, and/or psychiatric examination by a person of skill, for example, a physician, a psychologist, and/or psychiatrist and found to have a condition that can be diagnosed or treated by the compositions or methods disclosed herein. For example, “diagnosed with a CNS disease or disorder” or “diagnosed with a PNS disease or disorder” means having been subjected to a physical, psychological, and/or psychiatric examination by a person of skill, for example, a physician, a psychologist, and/or psychiatrist, and found to have a condition that can be diagnosed or can be treated by one or more compositions or methods disclosed herein.

As used herein, the term “treatment” can refer to the medical management of a subject with the intent to cure, ameliorate, stabilize, or prevent a disease, pathological condition, or disorder (such as, for example, a CNS or PNS disease or disorder). This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term can cover any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression or amelioration of the disease.

As used herein, the term “prevent” or “preventing” can refer to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed.

As used herein, the terms “administering” and “administration” refer to any method of providing a disclosed composition, complex, or a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In an aspect, a preparation can be administered therapeutically; that is, administered to treat an existing disease or condition. In an aspect, a preparation can be administered prophylactically; that is, administered for prevention of a disease or condition.

The term “contacting” as used herein can refer to bringing a disclosed composition, compound, or complex (such as, for example, one or more of the mediums disclosed herein) together with an intended target (such as, e.g., a cell or population of cells, a cell culture, a receptor, an antigen, or other biological entity) in such a manner that the disclosed composition, compound, or complex can affect the activity of the intended target (e.g., receptor, transcription factor, cell, population of cells, a cell culture, etc.), either directly (i.e., by interacting with the target itself), or indirectly (i.e., by interacting with another molecule, co-factor, factor, or protein on which the activity of the target is dependent). In an aspect, a disclosed composition or a disclosed medium can be contacted with a cell or population of cells, such as, for example, a population of neural progenitor cells or a population of neural crest stem cells. For example, a population of cells, such as neural progenitor cells, can be contacted with a disclosed medium (or disclosed mediums) by submerging the cells in a medium, coating the cells with a medium, dipping the cells in a medium, brushing the cells with a medium, bathing the cells in a medium, washing the cells in a medium. The skilled person is familiar with methods used to contact one or more mediums with cells, a population of cells, and/or a cell culture.

As used herein, the term “determining” can refer to measuring or ascertaining (i) an activity or an event, (ii) a quantity or an amount, (iii) a change in activity or an event, or (iv) a change in a quantity or an amount. Determining can also refer to measuring a change prevalence and/or incidence of an activity, or an event, or a trait, or a characteristic. For example, determining can refer to measuring or ascertaining the level of differentiation of a population of cells. The art is familiar with methods and techniques used to measure or ascertain (i) an activity or an event, (ii) a quantity or an amount, (iii) a change in activity or an event, (iv) a change in a quantity or an amount, or (v) a change in prevalence and/or incidence of an activity, or an event, or a trait, or a characteristic. For example, the art is well versed in the use of immunohistochemistry to identify, characterize, and quantify a particular cell type (e.g., a sensory neuron, a Schwann cell, a neural crest stem cell).

As used herein, the terms “effective amount” and “amount effective” can refer to an amount that is sufficient to achieve the desired result. For example, in an aspect, an effective amount of a disclosed composition or complex or agent is the amount effective to induce differentiation of a population of cells to a desired cell or a desired population of cells. For example, in an aspect, an amount effective is the amount of a disclosed composition or disclosed medium required to (i) induce differentiation of a population of neural progenitor cells to a population of neural crest cells, or (ii) induce differentiation of a population of neural progenitor cells to a population of sensory neurons, or (iii) induce differentiation of a population of neural crest stem cells to sensory neurons, or (iv) induce differentiation of a population of neural progenitor cells to Schwann cells.

A “therapeutically effective amount” can refer to an amount that is sufficient to achieve the desired therapeutic result or to have an effect on undesired symptoms (such as, for example, symptoms associated with a CNS or PNS disease or disorder), but is generally insufficient to cause adverse side effects. The specific therapeutically effective dose level for any particular subject will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a composition or complex at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”, that is, an amount effective for prevention of a disease or condition, such as a CNS or PNS disease or disorder including, but not limited to, those CNS and PNS diseases and disorders disclosed herein.

“Proliferation medium” can refer to supplemented AB2™ basal medium (Aruna Biomedical, Cat. No. hNP7011.3, see U.S. Pat. No. 6,200,806, which is herein incorporated by reference in its entirety for teachings regarding proliferation medium) comprising bFGF. Supplemented AB2 basal medium comprises ANS™ supplement (Aruna Biomedical, Cat. No. ANS7011.2), LIF, L-Glutamine, and Penicillin/Streptomycin.

“KSR medium” can refer to medium comprising SB435142 and Noggin. KSR medium can be prepared by supplementing Knockout DMEM (Invitrogen, Cat. 11330-032) with KSR (knockout serum replacement—Invitrogen, Cat. 10828-028), L-glutamine, penicillin/streptomycin, MEM, and β-mercaptoethanol. Noggin can bind to BMP molecules and prevents attachment to their corresponding receptors. SB431542 can inhibit the Lefty/Activin/TGF β pathways by blocking the phosphorylation of their respective receptors.

“N2B medium” can refer to a medium purchased at NeuralStem Inc. In an aspect, N2B can be equivalent to the N2 medium described in Lee et al., 2010. N2B medium can comprise distilled H₂O (985 ml) with DMEM/F12 powder, 1.55 g glucose, 2.00 g NaHCO₃, 25 mg insulin, 0.1 g apotransferrin, 30 nM sodium selenite, 100 μM putrescine, and 20 nM progesterone.

“KSR/N2B medium” can refer to medium comprising KSR medium and N2B medium.

“A2B™ basal neural medium” can refer to a medium engineered for the expansion and proliferation of hNP1 cells. A2B™ basal neural medium can allow neural cultures to maintain a stable karyotype over multiple passages.

It is understood that the compositions and mediums disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. METHODS

### i) Methods of Generating Sensory Neurons or Neural Crest Stem Cells

Disclosed herein are method of generating sensory cells or neural crest stem cells, comprising proliferating a population of neural progenitor cells; dissociating the population of proliferated neural progenitor cells; replating the population of dissociated neural progenitor cells; expanding the population of replated neural progenitor cells; and initiating differentiation of the population of expanded neural progenitor cells into sensory neurons or neural crest stem cells

In an aspect, a disclosed method of generating sensory cells or neural crest stem cells, can comprise promoting differentiation of the population of differentiating neural progenitor cells into sensory neurons. Disclosed herein is a use of a sensory neuron made using a disclosed method of generating sensory cells. Disclosed herein is a sensory neuron made using a disclosed method of generating sensory cells.

In an aspect, a disclosed method of generating sensory cells or neural crest stem cells can comprise dissociating the population of differentiating neural progenitor cells, replating the population of dissociated and differentiating neural progenitor cells, and promoting differentiation of the population of differentiating neural progenitor cells into neural crest stem cells. In an aspect, cells can be replated on glass coverslips pre-coated with poly-ornathine/laminin/fibronectin at a density of 200 cells/mm². In an aspect, replated cells can be maintained in N2B medium comprising bFGF and hEGF. In an aspect, N2B medium comprising bFGF and hEGR can be changed every two days. Disclosed herein is a use of a neural crest stem cell made using a disclosed method of generating neural crest stem cells. Disclosed herein is a neural crest stem cell made using a disclosed method of generating neural crest stem cell.

In an aspect, neural progenitor cells can be human or non-human neural progenitor cells. For example, in an aspect, neural progenitor cells can be hNP1 cells. In an aspect of a disclosed method of generating sensory cells or neural crest stem cells, feeder cells are not used.

In an aspect of a disclosed method of generating sensory cells or neural crest stem cells, proliferating the population of neural progenitor cells can comprise maintaining the cells in proliferation medium comprising bFGF. For example, in an aspect, proliferation medium can be changed every other day. In an aspect, proliferating the population of neural progenitor cells can comprise 3-5 days. In an aspect, proliferating population of neural progenitor cells can continue until the cells reach 100% confluence.

In an aspect of a disclosed method of generating sensory cells or neural crest stem cells, dissociating the population of neural progenitor cells can comprise manual dissociation. In an aspect, dissociating the population of neural progenitor cells can comprise manual dissociation, chemical dissociation, or combination thereof. Methods of dissociating cells are known to those skilled in the art.

In an aspect of a disclosed method of generating sensory cells or neural crest stem cells, replating the population of neural progenitor cells can comprise replating the cells onto glass coverslips at a density of 400 cells/mm². In an aspect, glass coverslips can be pre-coated with DETA, or poly-ornathine/laminin/fibronectin, or a combination thereof.

In an aspect of a disclosed method of generating sensory cells or neural crest stem cells, expanding the population of neural progenitor cells can comprise maintaining the population of cells in proliferation medium. In an aspect, cells can be maintained in proliferation medium for 2-3 days. In an aspect, expanding the population of neural progenitor cells can continue until the cells reach approximately 90% confluence.

In an aspect of a disclosed method of generating sensory cells or neural crest stem cells, initiating differentiation of the population of neural progenitor cells can comprise replacing proliferation medium with KSR medium; replacing KSR medium with N2B medium; and replacing N2B medium with a differentiation medium. In an aspect, KSR medium can comprise knockout DMEM, KSR, SB435142, Noggin, L-glutamine, MEM, penicillin/streptomycin, and beta-mercaptoethanol. In an aspect, replacing KSR medium with N2B medium can occur gradually. In an aspect, replacing KSR medium with N2B medium can comprise at least 10 days. For example, in an aspect, on Day 0, the medium can comprise 100% KSR medium. In an aspect, on Day 1, the medium can comprise 100% KSR medium. In an aspect, on Day 2, the medium can comprise 75% KSR medium and 25% N2B. In an aspect, on Day 3, the medium can comprise 75% KSR medium and 25% N2B. In an aspect, on Day 4, the medium can comprise 50% KSR medium and 50% N2B. In an aspect, on Day 5, the medium can comprise 50% KSR medium and 50% N2B. In an aspect, on Day 6, the medium can comprise 25% KSR medium and 75% N2B. In an aspect, on Day 7, the medium can comprise 25% KSR medium and 75% N2B. In an aspect, on Day 8, the medium can comprise 100% N2B. In an aspect, on Day 9, the medium can comprise 100% N2B. In an aspect, on Day 10, the medium can comprise and 100% N2B. In an aspect, the concentration of SB435142 and Noggin remain constant.

In an aspect of a disclosed method of generating sensory cells or neural crest stem cells, differentiation medium can comprise N2B medium comprising BDNF, L-ascorbic acid, GDNF, NGF, NT-3, cAMP, and Wnt-1. In an aspect, one-third of the differentiation medium can be changed every two days. In an aspect, cells can be maintained in the differentiation medium for at least 2 days.

### ii) Methods of Generating Sensory Neurons

Disclosed herein are methods of generating sensory neurons, comprising proliferating a population of neural progenitor cells; dissociating and replating the population of neural progenitor cells; expanding the population of neural progenitor cells; and initiating differentiation of the population of neural progenitor cells, whereby the population of neural progenitor cells differentiates into sensory neurons.

Disclosed herein are methods of generating sensory neurons, comprising proliferating a population of neural progenitor cells; dissociating the population of proliferated neural progenitor cells; replating the population of dissociated neural progenitor cells; expanding the population of replated neural progenitor cells; initiating differentiation of the population of expanded neural progenitor cells; and promoting differentiation of the population of differentiating neural progenitor cells, whereby the population of neural progenitor cells differentiates into sensory neurons.

In an aspect, neural progenitor cells can be human neural progenitor cells. In an aspect, neural progenitor cells can be non-human neural progenitor cells. In an aspect, neural progenitor cells can be hNP1 cells. In an aspect, hNP1 cells can be derived from WA09 cells. In an aspect, disclosed methods of generating sensory neurons do not comprise feeder cells. In an aspect, feeder cells are not used in the disclosed methods.

In an aspect of a disclosed method of generating sensory neurons, proliferating a population of neural progenitor cells can comprise maintaining the cells in proliferation medium. In an aspect, proliferation medium can be AB2 medium and can comprise bFGF. In an aspect, maintaining the population of neural progenitor cells in proliferation medium can comprise 3-5 days. For example, in an aspect, the population of neural progenitor cells can be maintained in proliferation medium for 3 days, or for 4 days, or for 5 days. In an aspect, the population of neural progenitor cells can be maintained in proliferation medium until the cells reach approximately or about 100% confluence. In an aspect, proliferation medium can be changed. In an aspect, proliferation medium can be changed repeatedly. For example, in an aspect, proliferation medium can be changed every other day.

In an aspect, proliferating the population of neural progenitor cells can comprise maintaining the cells in proliferation medium for 3-5 days, wherein the proliferation medium can be AB2 medium supplemented with bFGF, and wherein the proliferation medium can be changed every other day.

In an aspect of a disclosed method of generating sensory neurons, dissociating the population of neural progenitor cells can comprise manual dissociation, chemical dissociation, or a combination thereof.

In an aspect of a disclosed method of generating sensory neurons, replating the population of neural progenitor cells can comprise replating the cells onto glass coverslips. In an aspect, glass coverslips can be pre-coated with DETA, or poly-ornathine/laminin/fibronectin, or both DETA and poly-ornathine/laminin/fibronectin. In an aspect, replating the cells onto glass coverslips can be at a density of about 200 cells/mm² to about 600 cells/mm². In an aspect, replating the cells onto glass coverslips can be at a density of about 200 cells/mm², about 300 cells/mm², about 400 cells/mm², about 500 cells/mm², or about 600 cells/mm². In an aspect, replating the cells onto glass coverslips can be at a density of about 200 cells/mm².

In an aspect of a disclosed method of generating sensory neurons, expanding the population of neural progenitor cells can comprise maintaining the cells in proliferation medium. In an aspect, maintaining the cells in proliferation medium can comprise 1-6 days. In an aspect, maintaining the cells in proliferation medium can comprise 2-3 days. For example, in an aspect, the population of neural progenitor cells can be maintained in proliferation medium for at least 2 days or for at least 3 days. In an aspect, the population of neural progenitor cells can be maintained in proliferation medium until the cells reach approximately or about 90% confluence. In an aspect, the population of neural progenitor cells can be maintained in proliferation medium until the cells reach approximately or about 85-95% confluence. In an aspect, the population of neural progenitor cells can be maintained in proliferation medium until the cells reach approximately or about 80-85% confluence. In an aspect, the population of neural progenitor cells can be maintained in proliferation medium until the cells reach approximately or about 90-95% confluence.

In an aspect of a disclosed method of generating sensory neurons, initiating differentiation of the population of neural progenitor cells can comprise replacing proliferation medium with KSR medium; replacing KSR medium with N2B medium; and replacing N2B medium with differentiation medium. In an aspect, KSR medium can comprise knockout DMEM, KSR, SB435142, noggin, L-glutamine, MEM, penicillin/streptomycin, and beta-mercaptoethanol. In an aspect, differentiation medium can comprise N2B medium comprising BDNF, L-ascorbic acid, GDNF, NGF, NT-3, cAMP, and Wnt-1. In an aspect, medium can comprise KSR medium and N2B. In an aspect, replacing KSR medium with N2B medium can occur gradually. In an aspect, replacing KSR medium with N2B medium can comprise at least 10 days. For example, in an aspect, on Day 0, the medium can comprise 100% KSR medium. In an aspect, on Day 1, the medium can comprise 100% KSR medium. In an aspect, on Day 2, the medium can comprise 75% KSR medium and 25% N2B. In an aspect, on Day 3, the medium can comprise 75% KSR medium and 25% N2B. In an aspect, on Day 4, the medium can comprise 50% KSR medium and 50% N2B. In an aspect, on Day 5, the medium can comprise 50% KSR medium and 50% N2B. In an aspect, on Day 6, the medium can comprise 25% KSR medium and 75% N2B. In an aspect, on Day 7, the medium can comprise 25% KSR medium and 75% N2B. In an aspect, on Day 8, the medium can comprise 100% N2B. In an aspect, on Day 9, the medium can comprise 100% N2B. In an aspect, on Day 10, the medium can comprise and 100% N2B. In an aspect, the concentration of SB435142 and noggin in the medium can be constant. In an aspect, the concentration of SB435142 and noggin does not change.

In an aspect, some amount of the differentiation medium can be changed. In an aspect, the change in differentiation medium can be repeated (e.g., on some type of interval). In an aspect, approximately or about one-third of differentiation medium can be changed. In an aspect, approximately or about one-third of differentiation medium can be changed every two days. In an aspect, cells can be maintained in differentiation medium for at least 2-10 days. For example, in an aspect, cells can be maintained in differentiation medium for at least 2 days, at least 3 days, at least 4 days, at least 5 days, at least 6 days, at least 7 days, at least 8 days, at least 9 days, or at least 10 days. In an aspect, cells can be maintained in differentiation medium for 2 days.

Disclosed herein are methods of generating sensory neurons, comprising proliferating the population of neural crest stem cells; and initiating differentiation of the population of neural crest stem cells into sensory neurons. In an aspect, feeder cells are not used. In an aspect, proliferating the population of neural crest stem cells can comprise maintaining the cells in N2B medium comprising bFGF and hEGF. In an aspect, wherein N2B medium can be changed every other day. In an aspect, maintaining the cells in N2B medium can continue until the cells reach about 100% confluence. In an aspect of a method of generating sensory neurons, initiating differentiation of the population of neural crest stem cells can comprise replacing N2B medium with a differentiation medium. In an aspect, differentiation medium can comprise N2B medium comprising BDNF, L-ascorbic acid, GDNF, NGF, NT-3, cAMP, and Wnt-1. In an aspect, one-third of the medium can be changed every two days. In an aspect, the population of neural crest stem cells can be generated according to a disclosed method of generating neural crest stem cells. Disclosed herein is a use of a sensory neuron made using a disclosed method of generating sensory cells. Disclosed herein is a sensory neuron made using a disclosed method of generating sensory cells.

iii) Methods Generating Neural Crest Stem Cells

Disclosed here are methods of generating neural crest stem cells, comprising proliferating a population of neural progenitor cells; dissociating and replating the population of neural progenitor cells; expanding the population of neural progenitor cells; and initiating differentiation of the population of neural progenitor cells, whereby the population of neural progenitor cells differentiates into neural crest stem cells. In an aspect, neural progenitor cells can be human neural progenitor cells. In an aspect, neural progenitor cells can be non-human neural progenitor cells. In an aspect, neural progenitor cells can be hNP1 cells. In an aspect, hNP1 cells can be derived from WA09 cells. In an aspect, the disclosed methods of generating neural crest stem cells do not comprise feeder cells. In an aspect, feeder cells are not used in the disclosed methods.

Disclosed here are methods of generating neural crest stem cells, comprising proliferating a population of neural progenitor cells; dissociating the population of proliferated neural progenitor cells; replating the population of dissociated neural progenitor cells; expanding the population of replated neural progenitor cells; initiating differentiation of the population of expanded neural progenitor cells; dissociating the population of differentiating neural progenitor cells; and replating the population of dissociated and differentiating neural progenitor cells, whereby the population of neural progenitor cells differentiates into neural crest stem cells.

In an aspect of a disclosed method of generating neural crest stem cells, proliferating a population of neural progenitor cells can comprise maintaining the cells in proliferation medium. In an aspect, proliferation medium can be supplemented AB2 medium and can comprise bFGF. In an aspect, maintaining the population of neural progenitor cells in proliferation medium can comprise 3-5 days. For example, in an aspect, the population of neural progenitor cells can be maintained in proliferation medium for 3 days, or for 4 days, or for 5 days. In an aspect, the population of neural progenitor cells can be maintained in proliferation medium until the cells reach approximately or about 100% confluence. In an aspect, proliferation medium can be changed. In an aspect, proliferation medium can be changed repeatedly. For example, in an aspect, proliferation medium can be changed every other day. In an aspect, proliferating the population of neural progenitor cells can comprise maintaining the cells in proliferation medium, wherein the proliferation medium can comprise supplemented AB2 medium comprising bFGF, wherein the cells can be maintained in proliferation medium for 3-5 days, and wherein the proliferation medium can be changed every other day.

In an aspect of a disclosed method of generating neural crest stem cells, dissociating the population of neural progenitor cells can comprise manual dissociation, chemical dissociation, or combination thereof. In an aspect of a disclosed method of generating neural crest stem cells, replating the population of neural progenitor cells can comprise replating the cells onto glass coverslips. In an aspect, glass coverslips can be pre-coated with DETA, or poly-ornathine/laminin/fibronectin, or both DETA and poly-ornathine/laminin/fibronectin. In an aspect, replating the cells onto glass coverslips can be at a density of about 200 cells/mm² to about 600 cells/mm². In an aspect, replating the cells onto glass coverslips can be at a density of about 200 cells/mm², about 300 cells/mm², about 400 cells/mm², about 500 cells/mm², or about 600 cells/mm². In an aspect, replating the cells onto glass coverslips can be at a density of about 200 cells/mm².

In an aspect of a disclosed method of generating neural crest stem cells, expanding the population of neural progenitor cells can comprise maintaining the cells in proliferation medium. In an aspect, maintaining the cells in proliferation medium can comprise 1-6 days. In an aspect, maintaining the cells in proliferation medium can comprise 2-3 days. For example, in an aspect, the population of neural progenitor cells can be maintained in proliferation medium for at least 2 days or for at least 3 days. In an aspect, the population of neural progenitor cells can be maintained in proliferation medium until the cells reach approximately or about 90% confluence. In an aspect, the population of neural progenitor cells can be maintained in proliferation medium until the cells reach approximately or about 85-95% confluence. In an aspect, the population of neural progenitor cells can be maintained in proliferation medium until the cells reach approximately or about 80-85% confluence. In an aspect, the population of neural progenitor cells can be maintained in proliferation medium until the cells reach approximately or about 90-95% confluence.

In an aspect of a disclosed method of generating neural crest stem cells, initiating differentiation of the population of neural progenitor cells can comprise replacing proliferation medium with KSR medium; replacing KSR medium with N2B medium; and replacing N2B medium with differentiation medium. In an aspect, KSR medium can comprise knockout DMEM, KSR, SB435142, noggin, L-glutamine, MEM, penicillin/streptomycin, and beta-mercaptoethanol. In an aspect, differentiation medium can comprise N2B medium comprising BDNF, L-ascorbic acid, GDNF, NGF, NT-3, cAMP, and Wnt-1. In an aspect, medium can comprise KSR medium and N2B. In an aspect, replacing KSR medium with N2B medium can occur gradually. In an aspect, replacing KSR medium with N2B medium can comprise at least 10 days. For example, in an aspect, on Day 0, the medium can comprise 100% KSR medium. In an aspect, on Day 1, the medium can comprise 100% KSR medium. In an aspect, on Day 2, the medium can comprise 75% KSR medium and 25% N2B. In an aspect, on Day 3, the medium can comprise 75% KSR medium and 25% N2B. In an aspect, on Day 4, the medium can comprise 50% KSR medium and 50% N2B. In an aspect, on Day 5, the medium can comprise 50% KSR medium and 50% N2B. In an aspect, on Day 6, the medium can comprise 25% KSR medium and 75% N2B. In an aspect, on Day 7, the medium can comprise 25% KSR medium and 75% N2B. In an aspect, on Day 8, the medium can comprise 100% N2B. In an aspect, on Day 9, the medium can comprise 100% N2B. In an aspect, on Day 10, the medium can comprise 100% N2B. In an aspect, the concentration of SB43152 and Noggin in the medium can be constant. In an aspect, the concentration of SB435142 and noggin does not change.

In an aspect of a disclosed method of generating neural crest stem cells, initiating differentiation of the population of neural progenitor cells can comprise dissociating and replating the population of neural progenitor stem cells. In an aspect, dissociating can comprise manual dissociation, chemical dissociation, or a combination thereof. In an aspect, replating the population of neural progenitor cells can comprise replating the cells onto glass coverslips. In an aspect, glass coverslips can be pre-coated with DETA, or poly-ornathine/laminin/fibronectin, or both DETA and poly-ornathine/laminin/fibronectin. In an aspect, replating the cells onto glass coverslips can be at a density of about 50 cells/mm² to about 600 cells/mm². In an aspect, replating the cells onto glass coverslips can be at a density of about 50 cells/mm², about cells/mm², about 150 cells/mm², 200 cells/mm², about 300 cells/mm², about 400 cells/mm², about 500 cells/mm², or about 600 cells/mm².

In an aspect, replated cells can be maintained in N2B medium comprising bFGF and hEGF. In an aspect, the N2B medium comprising bFGF and hEGR can be changed. In an aspect, the N2B medium comprising bFGF and hEGF can be changed repeatedly. In an aspect, the N2B medium comprising bFGF and hEGR can be changed every two days.

### iv) Methods of Generating Sensory Neurons

Disclosed herein are methods of generating sensory neurons, comprising proliferating a population of neural crest stem cells; and initiating differentiation of the population of neural crest stem cells, whereby the population of neural crest stem cells differentiates into sensory neurons. In an aspect, neural progenitor cells can be human neural progenitor cells. In an aspect, neural progenitor cells can be non-human neural progenitor cells. In an aspect, the disclosed methods of generating sensory neurons do not comprise feeder cells. In an aspect, feeder cells are not used in the disclosed methods. In an aspect, the population of neural crest stem cells can be generated according to a method of generating neural crest stem cells disclosed herein. In an aspect, the population of neural crest stem cells can be purchased commercially.

In an aspect of a disclosed method of generating sensory neurons, proliferating the population of neural crest stem cells can comprise maintaining the cells in N2B medium comprising bFGF and hEGF. In an aspect, the population of neural crest stem cells can be maintained in N2B medium comprising bFGF and hEGF until the cells reach approximately or about 100% confluence. In an aspect, N2B medium comprising bFGF and hEGF can be changed. In an aspect, N2B medium comprising bFGF and hEGF can be changed repeatedly. For example, in an aspect, N2B medium comprising bFGF and hEGF can be changed every two days. In an aspect, proliferating the population of neural crest stem cells can comprise maintaining the cells in N2B medium comprising bFGF and hEGF until the cells reach approximately or about 100% confluence, wherein the N2B medium comprising bFGF and hEGF is changed every two days.

In an aspect of a disclosed method of generating sensory neurons, initiating differentiation of the population of neural crest stem cells can comprise replacing the N2B medium comprising bFGF and hEGF with differentiation medium. In an aspect, differentiation medium can comprise N2B medium comprising BDNF, L-ascorbic acid, GDNF, NGF, NT-3, cAMP, and Wnt-1. In an aspect, differentiation medium can be changed. In an aspect, differentiation medium can be changed repeatedly. In an aspect, one-third of differentiation medium can be changed every two days.

### v) Methods of Generating Schwann Cells

Disclosed herein are methods of generating Schwann cells, comprising proliferating the population of neural crest stem cells; and initiating the differentiation of the population of neural crest stem cells, whereby the population of neural crest stem cells differentiates into Schwann cells. In an aspect, the disclosed methods of generating Schwann cells do not comprise feeder cells. In an aspect, feeder cells are not used in the disclosed methods. In an aspect of a disclosed method of generating Schwann cells, the population of neural crest stem cells can be generated according to a disclosed method of generating the population of neural crest stem cells. In an aspect, the population of neural crest stem cells can be purchased commercially. In an aspect, neural progenitor cells can be human neural progenitor cells. In an aspect, neural progenitor cells can be non-human neural progenitor cells.

In an aspect of a disclosed method of generating Schwann cells, proliferating the population of neural crest stem cells can comprise maintaining the cells in N2B medium comprising bFGF and hEGF. In an aspect, the population of neural progenitor cells can be maintained in N2B medium comprising bFGF and hEGF until the cells reach approximately or about 100% confluence. In an aspect, N2B medium comprising bFGF and hEGF can be changed. In an aspect, N2B medium comprising bFGF and hEGF can be changed repeatedly. For example, in an aspect, proliferation medium can be changed every two days. In an aspect, proliferating the population of neural crest stem cells can comprise maintaining the cells in N2B medium comprising bFGF and hEGF until the cells reach approximately or about 100% confluence, wherein the N2B medium comprising bFGF and hEGF is changed every two days.

In an aspect of a disclosed method of generating Schwann cells, initiating the differentiation of the population of neural crest stem cells can comprise replacing the N2B medium comprising bFGF and hEGF with differentiation medium. In an aspect, differentiation medium can comprise N2B medium comprising CNTF, neuregulin, bFGF, and cAMP. In an aspect, medium can be changed. In an aspect, medium can be changed repeatedly. For example, in an aspect, one-third of medium can be changed every two days.

It is contemplated that each disclosed methods can further comprise additional steps, manipulations, and/or components. It is also contemplated that any one or more step, manipulation, and/or component can be optionally omitted from the invention. It is understood that a disclosed methods can be used to provide the disclosed compounds. It is also understood that the products of the disclosed methods can be employed in the disclosed methods of using.

Disclosed herein are methods of generating Schwann cells, comprising proliferating a population of neural crest stem cells; and initiating the differentiation of the population of neural crest stem cells into Schwann cells. In an aspect, feeder cells are not used. In an aspect of a disclosed method of generating Schwann cells, proliferating the population of neural crest stem cells can comprise maintaining the cells in N2B medium comprising bFGF and hEGF. In an aspect, N2B medium can be changed every two days. In an aspect, maintaining the cells in N2B medium can continue until the cells reach about 100% confluence. In an aspect of a disclosed method of generating sensory neurons, initiating the differentiation of the population of neural crest stem cells can comprise replacing the N2B medium with differentiation medium. In an aspect, differentiation medium can comprise N2B medium comprising CNTF, neuregulin, bFGF, and cAMP. In an aspect, one-third of the medium can be changed every two days. In an aspect, the population of neural crest stem cells can be generated using a disclosed method of generating neural crest stem cells. Disclosed herein is a use of a Schwann cell made using a disclosed method of generating Schwann cells. Disclosed herein is a Schwann cell made using a disclosed method of generating Schwann

### vi) Uses

a. Uses of Neural Crest Stem Cells

Disclosed herein are uses of neural crest stem cells made by a disclosed method of generating neural crest stem cells. For example, in an aspect, neural crest stem cells made by a disclosed method can be used as an in vitro source of sensory neurons. In an aspect, neural crest stem cells made by a disclosed method can be used as an in vitro source of Schwann cells. In an aspect, disclosed neural crest stem cells can be used in functional human (and non-human) disease models. In an aspect, disease models can be in vitro disease models. In an aspect, disclosed neural crest stem cells can be used in pathological studies. In an aspect, disclosed neural crest stem cells can be used in drug screening. In an aspect, disclosed neural crest stem cells can be used in regenerative medicine.

b. Uses of Sensory Neurons

Disclosed herein are uses of sensory neurons made by a disclosed method of generating sensory neurons. In an aspect, a disclosed method can comprise the population of neural progenitor cells. In an aspect, a disclosed method can comprise the population of neural crest stem cells. In an aspect, neural progenitor cells can be human neural progenitor cells. In an aspect, neural progenitor cells can be non-human neural progenitor cells. In an aspect, disclosed sensory neurons can be used in functional human (and non-human) disease models. In an aspect, disease models can be in vitro disease models. In an aspect, disclosed sensory neurons can be used in pathological studies. In an aspect, disclosed sensory neurons can be used in drug screening. In an aspect, disclosed sensory neurons can be used in regenerative medicine.

Sensory neurons made by a disclosed method can be used in regenerative medicine. For example, in an aspect, sensory neurons can be used during implantation into a subject. In an aspect, a subject has been diagnosed with or suffers from a disease or disorder that affects sensory neurons. In an aspect, implantation of sensory neurons generated by a disclosed method can treat or prevent disease or disorder that affects sensory neurons. In an aspect, following implantation of sensory neurons generated by a disclosed method, a subject can regain sensation or function or a subject can experience less pain, less numbness, or less discomfort, or a combination thereof.

Sensory neurons made by a disclosed method can be used in conjunction with a prosthetic device. For example, in an aspect, sensory neurons can be incorporated into a prosthetic device. In an aspect, a prosthetic device can be used by a subject. In an aspect, a subject has been diagnosed with or suffers from a disease or disorder that affects sensory neurons. In an aspect, a disclosed prosthetic device comprising sensory neurons generated by a disclosed method can assist a subject in performing tasks utilizing fine motor control. In an aspect, a prosthetic device comprising sensory neurons generated by a disclosed method can improve or enhance a subject's fine motor control. As known to the art, fine motor control is the coordination of muscles, bones, and nerves to produce small precise movements (e.g., fine motor control can be picking up a small item with the index finger and thumb).

Sensory neurons made by a disclosed method can be used to examine or characterize a mechanosensory complex. As known to the art, a vertebrate body has several conserved mechanisms for the transduction of mechanical forces to sensory neural impulses (i.e., mechanotransduction). These mechanisms include groups of mechanoreceptors, mainly cutaneous in nature, that respond to touch, pressure, and vibration as well as proprioceptors sensitive to changes in muscle length, stretch, and tension. The activity of this reflex mechanism assists in coordinating a diverse range of muscular activities including eye movement, respiration, and fine motor control. Both mechanoreceptors and proprioceptors are composed of specialized receptors innervated by a specific type of sensory neuron.

In an aspect, sensory neurons made by a disclosed method can be used to examine the role of a mechanosensory complex in the pathology of one or more muscular dystrophies. Muscular dystrophies refer to a group of inherited disorders that can involve muscle weakness and loss of muscle tissue. Muscular dystrophies are progressive in that these conditions worsen over time. Muscular dystrophies include, but are not limited to, the following: Becker muscular dystrophy, Duchenne muscular dystrophy, Emery-Dreifuss muscular dystrophy, Facioscapulohumeral muscular dystrophy, Limb-girdle muscular dystrophy, Myotonia congenita, and Myotonic dystrophy. Symptoms associated with various muscular dystrophies include intellectual disability, muscle weakness, delayed development of muscle motor skills, difficulty using one or more muscle groups, drooling, eyelid drooping, frequent falls, loss of strength in a muscle or group of muscles as an adult, loss in muscle size, and difficulty in walking.

Sensory neurons made by a disclosed method can be used in several in vitro platforms or models. For example, in an aspect, sensory neurons made by a disclosed method can be used in an in vitro platform to characterize and/or examine one or more diseases or disorders, such as a CNS or PNS disease or disorder, including those diseases and disorders disclosed herein. For example, in an aspect, sensory neurons made by a disclosed method can be used in an in vitro platform or model to characterize and/or examine one or more of the following diseases and disorders: peripheral neuropathy, neuropathic pain, peripheral nerve regeneration, leprosy, and spasticity inducing diseases like Parkinson's disease. In an aspect, a disease or disorder can be peripheral neuropathy. In an aspect, a disease or disorder can be neuropathic pain. In an aspect, a disease or disorder can be peripheral nerve regeneration. In an aspect, a disease or disorder can be leprosy. In an aspect, a disease or disorder can be spasticity inducing diseases like Parkinson's disease.

Sensory neurons made by a disclosed method can be used in the development of in vitro models of drug screening. In an aspect, drug screening can be used for a disease or disorder that affects sensory neurons. In an aspect, drug screening can be used one or more muscular dystrophies. In an aspect, drug screening can be used with peripheral neuropathy, neuropathic pain, peripheral nerve regeneration, leprosy, and spasticity inducing diseases like Parkinson's disease.

c. Uses of Schwann Cells

Disclosed herein are uses of Schwann cell generated by a disclosed method for generating Schwann cells. In an aspect, disclosed Schwann cells can be used in functional human (and non-human) disease models. In an aspect, disease models can be in vitro disease models. In an aspect, disclosed Schwann cells can be used in pathological studies. In an aspect, disclosed Schwann cells can be used in drug screening. In an aspect, disclosed Schwann cells can be used in regenerative medicine.

For example, Schwann cells made by a disclosed method can be used in regenerative medicine. For example, in an aspect, Schwann cells can be used during implantation into a subject. In an aspect, a subject has been diagnosed with or suffers from a disease or disorder that affects Schwann cells. In an aspect, implantation of Schwann cells generated by a disclosed method can treat or prevent disease or disorder that affects Schwann cells. In an aspect, following implantation of Schwann cells generated by a disclosed method, a subject can regain sensation or function or a subject can experience less pain, less numbness, less discomfort, or a combination thereof.

Schwann cells made by a disclosed method can be used in conjunction with a prosthetic device. For example, in an aspect, Schwann cells can be incorporated into a prosthetic device. In an aspect, a prosthetic device can be used by a subject. In an aspect, a subject has been diagnosed with or suffers from a disease or disorder that affects Schwann cells. In an aspect, a disclosed prosthetic device comprising Schwann cells generated by a disclosed method can assist a subject in performing tasks utilizing fine motor control. In an aspect, a prosthetic device comprising Schwann cells generated by a disclosed method can improve or enhance a subject's fine motor control. As known to the art, fine motor control is the coordination of muscles, bones, and nerves to produce small precise movements (e.g., fine motor control can be picking up a small item with the index finger and thumb).

Schwann cells made by a disclosed method can be used to examine or characterize a mechanosensory complex. As known to the art, a vertebrate body has several conserved mechanisms for the transduction of mechanical forces to sensory neural impulses (i.e., mechanotransduction). These mechanisms include groups of mechanoreceptors, mainly cutaneous in nature, that respond to touch, pressure, and vibration as well as proprioceptors sensitive to changes in muscle length, stretch, and tension. The activity of this reflex mechanism assists in coordinating a diverse range of muscular activities including eye movement, respiration, and fine motor control. Both mechanoreceptors and proprioceptors are composed of specialized receptors innervated by a specific type of sensory neuron.

In an aspect, Schwann cells made by a disclosed method can be used to examine the role of a mechanosensory complex in the pathology of one or more muscular dystrophies. Muscular dystrophies refer to a group of inherited disorders that can involve muscle weakness and loss of muscle tissue. Muscular dystrophies are progressive in that these conditions worsen over time. Muscular dystrophies include, but are not limited to, the following: Becker muscular dystrophy, Duchenne muscular dystrophy, Emery-Dreifuss muscular dystrophy, Facioscapulohumeral muscular dystrophy, Limb-girdle muscular dystrophy, Myotonia congenita, and Myotonic dystrophy. Symptoms associated with various muscular dystrophies include intellectual disability, muscle weakness, delayed development of muscle motor skills, difficulty using one or more muscle groups, drooling, eyelid drooping, frequent falls, loss of strength in a muscle or group of muscles as an adult, loss in muscle size, and difficulty in walking.

Schwann cells made by a disclosed method can be used in several in vitro platforms or models. For example, in an aspect, Schwann cells made by a disclosed method can be used in an in vitro platform to characterize and/or examine one or more diseases or disorder. For example, in an aspect, Schwann cells made by a disclosed method can be used in an in vitro platform or model to characterize and/or examine one or more of the following diseases and disorders: peripheral neuropathy, neuropathic pain, peripheral nerve regeneration, leprosy, and spasticity inducing diseases like Parkinson's disease. In an aspect, a disease or disorder can be peripheral neuropathy. In an aspect, a disease or disorder can be neuropathic pain. In an aspect, a disease or disorder can be peripheral nerve regeneration. In an aspect, a disease or disorder can be leprosy. In an aspect, a disease or disorder can be spasticity inducing diseases like Parkinson's disease.

Schwann cells made by a disclosed method can be used in the development of in vitro models of drug screening. In an aspect, drug screening can be used for a disease or disorder that affects sensory neurons. In an aspect, drug screening can be used for one or more muscular dystrophies. In an aspect, drug screening can be used for peripheral neuropathy, neuropathic pain, peripheral nerve regeneration, leprosy, and spasticity inducing diseases like Parkinson's disease.

### C. COMPOSITIONS

Disclosed herein are sensory neurons made by a disclosed method of generating sensory neurons, wherein the method comprises the population of neural progenitor cells. Disclosed herein are sensory neurons made by a disclosed method of generating sensory neurons, wherein the method comprises the population of neural crest stem cells. Disclosed herein are neural crest stem cells made by a disclosed method of generating neural crest stem cells. Disclosed herein are Schwann cells made by a disclosed method for generating Schwann cells.

Disclosed herein are mediums for use in one or more of the disclosed methods.

vii) Kits

Disclosed herein are kit, comprising a population of neural progenitor cells; and instructions for differentiating the population of neural progenitor cells into sensory neurons. In an aspect, neural progenitor cells can be human neural progenitor cells. In an aspect, neural progenitor cells can be non-human neural progenitor cells. In an aspect, neural progenitor cells can be hNP1 cells. In an aspect, hNP1 cells can be derived from WA09 cells.

In an aspect, a disclosed kit can comprise one or more mediums, two or more medium, three or more mediums, or four or more mediums. In an aspect, mediums can comprise a first medium, a second medium, a third medium, and a fourth medium. In an aspect, a disclosed kit comprises a first medium, a second medium, a third medium, and a fourth medium.

In an aspect, a first medium can be used during proliferation of the population of neural progenitor cells. In an aspect, a first medium can be used during expansion of the population of neural progenitor cells. In an aspect, a first medium can be used during proliferation of the population of neural progenitor cells and during expansion of the population of neural progenitor cells. In an aspect, a first medium can comprise supplemented AB2 basal medium comprising bFGF. In an aspect, a second medium can be used during induction of differentiation of the population of neural progenitor cells.

In an aspect, a second medium can comprise knockout DMEM, KSR, L-glutamine, MEM, penicillin/streptomycin, beta-mercaptoethanol, SB435142, and noggin. In an aspect, knockout DMEM and KSR (Knockout Serum Replacement) can be purchased commercially from, for example, a company such as Invitrogen.

In an aspect, a third medium can be used during promotion of differentiation of the population of neural progenitor cells. In an aspect, a third medium can comprise N2B medium. In an aspect, a third medium can comprise N2B medium and N2B can comprise Noggin and SB431542. In an aspect, N2B medium can be purchased commercially from, for example, a company such as NeuralStem Inc.

In an aspect, a fourth medium can be used promotion of differentiation of the population of neural progenitor cells. In an aspect, a fourth medium can comprise N2B medium comprising BDNF, L-ascorbic acid, GDNF, NGF, NT-3, cAMP, and Wnt-1. In an aspect, N2B medium can be purchased commercially from, for example, a company such as NeuralStem Inc.

In an aspect, a disclosed kit can comprise glass coverslips. In an aspect, glass coverslips can be pre-coated with DETA, poly-ornathine/laminin/fibronectin, or both DETA and poly-ornathine/laminin/fibronectin.

Disclosed herein are kits comprising a population of neural progenitor cells; and instructions for differentiating the population of neural progenitor cells into neural crest stem cells. In an aspect, neural progenitor cells can be human neural progenitor cells. In an aspect, neural progenitor cells can be non-human neural progenitor cells. In an aspect, neural progenitor cells can be hNP1 cells. In an aspect, hNP1 cells can be derived from WA09 cells.

In an aspect, a disclosed kit can comprise one or more mediums, two or more medium, three or more mediums, or four or more mediums. In an aspect, mediums can comprise a first medium, a second medium, a third medium, and a fourth medium. In an aspect, a disclosed kit comprises a first medium, a second medium, a third medium, and a fourth medium.

In an aspect, first medium can be used during proliferation of the population of neural progenitor cells. In an aspect, a first medium can be used during expansion of the population of neural progenitor cells. In an aspect, a first medium can be used during proliferation of the population of neural progenitor cells and during expansion of the population of neural progenitor cells. In an aspect, a first medium can comprise supplemented AB2 basal medium comprising bFGF.

In an aspect, a second medium can be used during induction of differentiation of the population of neural progenitor cells. In an aspect, a second medium can comprise knockout DMEM, KSR, L-glutamine, MEM, penicillin/streptomycin, beta-mercaptoethanol, SB435142, and noggin. In an aspect, knockout DMEM and KSR (Knockout Serum Replacement) can be purchased commercially from, for example, a company such as Invitrogen.

In an aspect, a third medium can be used during promotion of differentiation of the population of neural progenitor cells. In an aspect, a third medium can comprise N2B medium. In an aspect, a third medium can comprise N2B medium and N2B can comprise Noggin and SB431542. In an aspect, N2B medium can be purchased commercially from, for example, a company such as NeuralStem Inc.

In an aspect, a fourth medium can be used during promotion of proliferation of the population of neural crest stem cells. In an aspect, a fourth medium can comprise N2B medium comprising bFGF and hEGF.

In an aspect, a disclosed kit can comprise glass coverslips. In an aspect, glass coverslips can be pre-coated with DETA, poly-ornathine/laminin/fibronectin, or both DETA and poly-ornathine/laminin/fibronectin.

Disclosed herein are kits comprising a population of neural crest stem cells; and instructions for differentiating the population of neural crest stem cells into sensory neuron, Schwann cells, or both. In an aspect, the population of neural crest stem cells can be generated according to a disclosed method of generating the population of neural crest stem cells. In an aspect, the population of neural crest stem cells can be purchased commercially. In an aspect, neural progenitor cells can be human neural progenitor cells. In an aspect, neural progenitor cells can be non-human neural progenitor cells.

In an aspect, a disclosed kit can comprise one or more mediums, two or more medium, three or more mediums, or four or more mediums. In an aspect, mediums can comprise a first medium, a second medium, and a third medium. In an aspect, a disclosed kit comprises a first medium, a second medium, and a third medium.

In an aspect, a first medium can be used during proliferation of the population of neural crest stems cells. In an aspect, a first medium can comprise N2B medium comprising bFGF and hEGF. In an aspect, a second medium can be used during differentiation of the population of neural crest stems cells into sensory neurons.

In an aspect, a second medium can comprise N2B medium comprising BDNF, L-ascorbic acid, GDNF, NGF, NT-3, cAMP, and Wnt-1. In an aspect, a third medium can be used during differentiation of the population of neural crest stem cells into Schwann cells.

In an aspect, a third medium can comprise N2B medium comprising CNTF, neuregulin, bFGF, and cAMP.

In an aspect, a disclosed kit can comprise glass coverslips. In an aspect, glass coverslips can be pre-coated with DETA, poly-ornathine/laminin/fibronectin, or both DETA and poly-ornathine/laminin/fibronectin.

Disclosed herein are methods of generating Schwann cells, comprising proliferating a population of neural crest stem cells; and initiating the differentiation of the population of neural crest stem cells into Schwann cells. In an aspect, feeder cells are not used. In an aspect of a disclosed method of generating Schwann cells, proliferating the population of neural crest stem cells can comprise maintaining the cells in N2B medium comprising bFGF and hEGF. In an aspect, N2B medium can be changed every two days. In an aspect, maintaining the cells in N2B medium can continue until the cells reach about 100% confluence. In an aspect of a disclosed method of generating sensory neurons, initiating the differentiation of the population of neural crest stem cells can comprise replacing the N2B medium with differentiation medium. In an aspect, differentiation medium can comprise N2B medium comprising CNTF, neuregulin, bFGF, and cAMP. In an aspect, one-third of the medium can be changed every two days. In an aspect, the population of neural crest stem cells can be generated using a disclosed method of generating neural crest stem cells. Disclosed herein is a use of a Schwann cell made using a disclosed method of generating Schwann cells. Disclosed herein is a Schwann cell made using a disclosed method of generating Schwann cell.

Disclosed herein are kits, comprising a population of neural progenitor cells; and instructions for differentiating the population of neural progenitor cells into sensory neurons. In an aspect, the neural progenitor cells can be human or non-human neural progenitor cells. In an aspect, the neural progenitor cells can be hNP1 cells. In an aspect, a disclosed kit can comprise one or more mediums. For example, in an aspect, the one or more mediums can comprise a first medium, wherein the first medium can be used during proliferation of the population of neural progenitor cells and wherein the first medium can be used during expansion of the population of neural progenitor cells; a second medium, wherein the second medium can be used during induction of differentiation of the population of neural progenitor cells; a third medium, wherein the third medium can be used during promotion of differentiation of the population of neural progenitor cells; and a fourth medium, wherein the fourth medium can be used during promotion of differentiation of the population of neural progenitor cells. In an aspect, the first medium can comprise AB2 basal medium comprising bFGF. In an aspect, the second medium can comprise knockout DMEM, KSR, L-glutamine, MEM, penicillin/streptomycin, beta-mercaptoethanol, SB435142, and noggin. In an aspect, the third medium can comprise N2B medium. In an aspect, the fourth medium can comprise N2B medium comprising BDNF, L-ascorbic acid, GDNF, NGF, NT-3, cAMP, and Wnt-1. In an aspect, a disclosed kit can comprise glass coverslips, wherein the glass coverslips are pre-coated with DETA, poly-ornathine/laminin/fibronectin, or a combination thereof.

Disclosed here are kits, comprising a population of neural progenitor cells; and instructions for differentiating the population of neural progenitor cells into neural crest stem cells. In an aspect, the neural progenitor cells can be human or non-human neural progenitor cells. In an aspect, the neural progenitor cells can be neural progenitor cells. In an aspect, the neural progenitor cells can be hNP1 cells. In an aspect, a disclosed kit can comprise one or more mediums. In an aspect, the one or more mediums can comprise a first medium, wherein the first medium can be used during proliferation of the population of neural progenitor cells and wherein the first medium can be used during expansion of the population of neural progenitor cells; a second medium, wherein the second medium can be used during induction of differentiation of the population of neural progenitor cells; a third medium, wherein the third medium can be used during promotion of differentiation of the population of neural progenitor cells; and a fourth medium, wherein the fourth medium can be used during promotion of differentiation of the population of neural progenitor cells. In an aspect, the first medium can comprise AB2 basal medium comprising bFGF. In an aspect, the second medium can comprise knockout DMEM, KSR, L-glutamine, MEM, penicillin/streptomycin, beta-mercaptoethanol, SB435142, and noggin. In an aspect, the third medium can comprise N2B medium. In an aspect, the fourth medium can comprise N2B medium comprising bFGF and hEGF. In an aspect, a discoed kit can comprise glass coverslips, wherein the glass coverslips can be pre-coated with DETA, or poly-ornathine/laminin/fibronectin, or a combination thereof.

Disclosed here are kits, comprising a population of neural crest stem cells; and instructions for differentiating the population of neural crest stem cells into sensory neuron, Schwann cells, or both. In an aspect, the population of neural crest stem cells can be generated according to a disclosed method of generating neural crest stem cells. In an aspect, a disclosed kit can comprise one or more mediums. In an aspect, the one or more mediums can comprise a first medium, wherein the first medium can be used during proliferation of the population of neural crest stems cells; a second medium, wherein the second medium can be used during differentiation of the population of neural crest stems cells into sensory neurons; and a third medium, wherein the third medium can be used during differentiation of the population of neural crest stem cells into Schwann cells. In an aspect, the first medium can comprise N2B medium comprising bFGF and hEGF. In an aspect, the second medium can comprise N2B medium comprising BDNF, L-ascorbic acid, GDNF, NGF, NT-3, cAMP, and Wnt-1. In an aspect, the third medium can comprise N2B medium comprising CNTF, neuregulin, bFGF, and cAMP. In an aspect, a disclosed kit can comprise glass coverslips, wherein the glass coverslips can be pre-coated with DETA, or poly-ornathine/laminin/fibronectin, or a combination thereof.

### D. EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

The experiments described herein provide an efficient means for generating populations of terminally differentiated sensory neurons and Schwann cells. Damage to the sensory neurons can cause symptoms ranging from numbness to pain to a lack of coordination. Schwann cells are the myelinating cells of the PNS and are one of the major cell groups that provide trophic support to peripheral neurons as well. The sensory neurons and Schwann cells disclosed herein provide invaluable tools for the characterization and treatment of several disease and disorders.

### i) Experimental Materials and Methods

a. DETA Surface Modification

DETA (a self-assembled monolayer (SAM) of N-1(3-(trimethoxysilyl)propyl) diethylenetriamine) supports neuronal growth as well as or better than biological surfaces. (Das et al., 2003; Hickman et al., 1994). DETA also supports the growth of both embryonic and adult MNs. (Das et al., 2005; Das et al., 2007). For DETA modification, glass coverslips (6661F52, 22×22 mm No. 1; Thomas Scientific, Swedesboro, N.J., USA) were cleaned using HCl/methanol (1:1) for at least 2 hours, rinsed with deionized water, soaked in concentrated H₂SO₄ for at least 2 hours and rinsed again with deionized water. Coverslips were boiled in nanopure water and then oven dried. The DETA (T2910KG; United Chemical Technologies Inc., Bristol, Pa., USA) film was formed by the reaction of the cleaned surfaces with a 0.1% (v/v) mixture of the organosilane in freshly distilled toluene (T2904; Fisher, Suwanne, Ga., USA). The DETA coated coverslips were heated to ˜80° C., cooled to room temperature (RT), rinsed with toluene, reheated to approximately the same temperature, and then cured for at least 2 hours at 110° C. Surfaces were characterized by contact angle and X-ray photoelectron spectroscopy as described previously. (Das et al., 2003, Das et al., 2005, Das et al., 2007).

b. Culture of Human Progenitor Cells

Human neural progenitor cells, STEMEZ™ hNP1, were obtained from Neuromics (Edina, Minn.). The cells were derived from the human embryonic stem cell line WA09 (H9), which have been differentiated past the point of requiring feeder cells or expensive feeder free systems. These cells can be cultured as adherent monolayers. The cells were expanded and maintained as described in the STEMEZ™ hNP1 expansion kit, which is incorporated herein by reference in its entirety for it teachings regarding the expansion and maintenance of these cells. Briefly, the cells were proliferated on a Matrigel-coated surface in proliferation medium (supplemented AB2™ basal medium from Neuromics containing 20 ng/mL bFGF (R&D system, Cat. No. 234-FSE-025/CF)). The Matrigel-coated surface was prepared by incubating BD Matrigel™ (1:200 diluted in DMEM—BD Biosciences, Cat. No. 356234) with the cell culture surface for 1 hour at room temperature, rinsing briefly with DMEM, and using immediately for cell plating. The cells were plated at ˜75% confluence and harvested for passaging by manual dissociation when ˜100% confluence was reached. At various passages, the cells were frozen in growth medium, plus 10% dimethyl sulfoxide (DMSO), at 1×10⁶ cells/mL using a programmable freezer. The frozen cells were then stored in liquid nitrogen. The product information provided by Neuromics specifies that their cells can be expanded through ten (10) passages before any genotypic monitoring was necessary (http://www.neuromics.com/). In the experiments described herein, passage 9 or 10 cells were used.

Table 1 provides the compositions (and potential commercial sources) of several of the mediums disclosed and utilized herein.

| | | | |
|---|---|---|---|
| Full Name | Conc. | Company | Cat. No. |

Neural Crest Stem Cell Differentiation Media or KSR/N2B Medium

| | | | | |
|---|---|---|---|---|
| Noggin | 500 | ng/mL | R&D | 6057- |
| | | | | NG/CF |
| SB431542 | 10 | μM | Tocris | 1614 |
| | | | Bioscience | |
| Wnt1 | 10 | ng/mL | Sigma | SRP4754 |

Neural Crest Stem Cell Growth Media in N2B Medium

| | | | | |
|---|---|---|---|---|
| Basic Fibroblast Growth | 50 | ng/mL | R&D | 234- |
| Factor (bFGF) | | | | FSE/CF |
| Human Epidermal | 10 | ng/mL | Invitrogen | PHG0314 |
| Growth Factor (hEGF) | | | | |

hSN Differentiation Media

| | | | | |
|---|---|---|---|---|
| N2B | | | NeuralStem | |
| | | | Inc. | |
| Brain-Derived Neurotrophic | 10 | ng/mL | Cell Sciences | CRB600B |
| Factor (BDNF) | | | | |
| Neurotrophin-3 (NT-3) | 10 | ng/mL | Cell Sciences | CRN500B |
| Glial Cell-Derived | 10 | ng/mL | Cell Sciences | CRG400B |
| Neurotrophic Factor | | | | |
| (GDNF) | | | | |
| Nerve Growth Factor | 10 | ng/mL | R&D | 256-GF |
| (NGF) | | | | |
| Adenosine 3′,5′-cyclic | 0.5 | μM | Sigma | A-9501 |
| Monophosphate (cAMP) | | | | |
| L-Ascorbic Acid | 200 | μM | Sigma | 396-HB |

c. Induction of Sensory Neurons

The overall induction protocol consisted of three steps: proliferation, expansion, and differentiation. For proliferation, 1×10⁶ cells were seeded into a 35 mm cell culture dish, pre-coated with BD Matrigel, and maintained in the proliferation medium (supplemented AB2 medium comprising bFGF (20 ng/mL)). The proliferation medium was changed every other day. The cells were proliferated for 3 to 5 days until 100% confluence was reached. Next, they were manually dissociated, re-plated onto glass coverslips pre-coated with DETA followed by poly-ornathine/laminin/fibronectin (Lee et al., 2010) at a density of 400 cells/mm². The cells were then expanded in proliferation medium for 2 to 3 days to enable ˜90% confluence before induction.

To initiate sensory neuron differentiation, the medium was replaced with KSR medium that contained 10 μM SB435142 and 500 ng/mL noggin. KSR medium was prepared by supplementing 800 mL Knockout DMEM (Invitrogen, Cat. No. 11330-032) with 150 mL Knockout Serum Replacement or KSR (Invitrogen, Cat. No. 10828-028), 10 mL L-Glutamine (Invitrogen, Cat. No. 21051-016), 10 mL Penicillin/Streptomycin (100X, Invitrogen, Cat. No. 15070-063), 10 mL 10 mM MEM (100X, nonessential amino acids, Invitrogen, Cat. No. 11140-050) and 1 mL β-mercaptoethanol (1,000X, Invitrogen, Cat. No. 21985-023). For two days, the cells were only exposed only to the KSR medium.

Then, to feed the cells during differentiation, the medium was replaced and gradually switched from KSR medium to N2B medium (NeuralStem Inc.) according to the following schedule: day 2 (75% KSR/25% N2B), day 4 (50% KSR/50% N2B), day 6 (25% KSR/75% N2B), days 8 and 10 (0% KSR/100% N2B). However, the concentration of SB435142 (10 μM) and Noggin (500 ng/mL) remained constant throughout the experiments. On days 0 and 1, the medium was 100% KSR. On days 8-11, the medium was 100% N2B.

Starting with day 12, the cells were fed with a differentiation medium by changing one-third of the medium every 2 days. The differentiation medium comprised N2B medium supplemented with BDNF (10 ng/mL), L-ascorbic Acid (200 μM), GDNF (10 ng/mL), NGF (10 ng/mL), NT-3 (10 ng/mL), cAMP (20 μM), and Wnt-1 (10 ng/mL). Then, on Day 14, just two days after first receiving the differentiation medium, the cells were analyzed by immunocytochemistry and electrophysiology.

d. Isolation of Neural Crest Stem Cells (NCSCs)

In a second set of experiments, on day 10 of the above-described timeline (i.e., medium was 100% N2B), cells were harvested by manual dissociation. The harvested cells were re-plated onto glass coverslips pre-coated with poly-ornathine/laminin/fibronectin at a density of 200 cells/mm². The cells were maintained in N2B medium containing bFGF (20 ng/mL) and hEGF (10 ng/mL). The cells were fed every 2 days with the same medium until the cells reached confluence. Then, the cells were harvested by manual dissociation and passaged onto coverslips with an identical surface treatment in the same medium (i.e., N2B medium containing bFGF and hEGF). At various passages, the cells were frozen in the N2B medium plus 10% Dimethyl Sulfoxide (DMSO) at 1×10⁶ cells/mL using a programmable freezer. The frozen cells were then stored in liquid nitrogen.

e. Differentiation of Neural Crest Stem Cells (NCSCs)

The NCSCs described above proliferated until near confluence in N2B medium containing bFGF (20 ng/mL) and hEGF (10 ng/mL). During proliferation, the cells were fed every 2 days with the same N2B medium. Then, cells were subjected to two separate differentiation processes—differentiation into (1) sensory neurons or (2) Schwann cells.

First, to induce differentiation of the NCSCs into sensory neurons, the cells were fed with differentiation medium (i.e., N2B medium comprising BDNF (10 ng/mL), L-ascorbic Acid (200 μM), GDNF (10 ng/mL), NGF (10 ng/mL), NT-3 (10 ng/mL), cAMP (20 μM), and Wnt-1 (10 ng/mL)) by changing one-third of the medium every 2 days. Fifteen (15) days later, the cells were fixed for immunocytochemistry.

Second, to induce differentiation of the NCSCs into Schwann cells, the cells were similarly fed by changing one-third of the medium every 2 days using a Schwann cell medium as described in Lee et al., 2010, which is incorporated herein by reference in its entirety, with slight modifications. Briefly, the Schwann cell medium consisted of N2B medium supplemented with CNTF (10 ng/mL), Neuregulin (20 ng/mL), bFGF (10 ng/mL) and cAMP (5 μM). Fifteen (15) days later, the cells were fixed for immunocytochemistry.

f. Immunocytochemistry and Microscopy

Cells were fixed in freshly prepared 4% paraformaldehyde for 15 minutes. Cells were washed twice in phosphate buffered saline (PBS) (pH 7.2, w/o Mg²⁺, Ca²⁺) for 10 minutes each time at room temperature and then permeabilized with 0.1% triton X-100/PBS for 15 minutes. Non-specific binding sites were blocked with 5% donkey serum plus 0.5% BSA in PBS (blocking buffer) for 45 minutes at room temperature. Cells were incubated with primary antibodies overnight at 4° C. After being washed with PBS three times for 10 minutes each, the cells were incubated with secondary antibodies for 2.5 hours at room temperature. The cells were then washed with PBS three times for 10 minutes each time and mounted with Vectashield with 4′-6-Diamidino-2-Phenylindole (DAPI) (Vector Laboratories, Inc.).

Primary antibodies used herein were: rabbit-anti-nestin (Chemicon, 1:200), rabbit-anti βIII (Sigma, 1:1000), rabbit-anti-SOX1 (Millipore, 1:100), goat-anti-peripherin (Santa Cruz Biotech, 1:25), mouse-anti-HNK1 (Sigma, 1:400), rabbit-anti-Brn3a (millipore, 1:1000), rabbit-anti-S100 (sigma, 1:400), rabbit-anti-MASH1 (Abcam, 1:500), guinea pig-anti-parvalbumin (millipore, 1:100), vGluT1 (Millipore, 1:400), rat-anti-substance P (Millipore, 1:50), rabbit-anti-P75 (Millipore, 1:200), and mouse-anti-GFAP (Chemicon, 1:150). Secondary antibodies used herein were as follows: donkey-anti-goat-594, donkey-anti-goat-488, donkey-anti-mouse-488, donkey-anti-mouse-594, donkey-anti-rabbit-594, donkey-anti-rabbit-488, and donkey-anti-guinea Pig-488. All secondary antibodies were from Invitrogen and used at 1:250 dilution. All antibodies were diluted in Blocking Buffer.

g. Electrophysiological Recordings

For electrophysiological recordings, the hNP1-derived sensory neurons were plated and differentiated on glass coverslips coated with DETA. The cells were characterized in cultures differentiated for 2 to 4 weeks using whole-cell patch-clamp recording techniques (Das et al., 2003, Liu et al., 2008). The recordings were performed in a recording chamber located on the stage of a Zeiss Axioscope 2FS Plus upright microscope (Gao et al., 1998). Sensory neurons were visually identified under an infrared DIC-video microscope. The large round bipolar or pseudo-unipolar cells (15-25 μm diameter) with bright illuminance in the culture were tentatively identified as sensory neurons. Patch pipettes with a resistance of 5-10 MΩ were made from borosilicate glass (BF 150-86-10; Sutter, Novato, Calif.) with a Sutter P97 pipette puller (Sutter Instrument Company). Current-clamp and voltage-clamp recordings were performed utilizing a Multiclamp 700A amplifier (Axon, Union City, Calif.). The pipette (intracellular) solution contained (in mM) K-gluconate 140, MgCl₂ 2, Na₂ATP 2, Phosphocreatine 5, Phosphocreatine kinase 2.4 mg, Hepes 10; pH 7.2.

After the formation of a GΩ seal and membrane puncture, cell capacitance was compensated. The series resistance was typically <23 MΩ, and it was compensated >60% using the amplifier circuitry. Signals were filtered at 3 kHz and sampled at 20 kHz using a Digidata 1322A interface (Axon instrument). Data recording and analysis were performed with pClamp8 software (Axon instrument). Membrane potentials were corrected by subtraction of a 15 mV tip potential, which was calculated using Axon's pClamp8 program. Membrane resistance and capacitance were calculated using 50 ms voltage steps from −85 to −95 mV without any whole-cell or series resistance compensation. The resting membrane potential and depolarization-evoked action potentials (APs) were recorded in current-clamp mode. Depolarization-evoked inward and outward currents were examined in voltage-clamp mode.

h. Quantification of Results

Immunocytochemical characterization and quantification of the differentiated cultures utilized a minimum of six (6) pictures taken from randomly chosen, un-clustered areas on each coverslip. The ratio of marker-positive to DAPI-positive cells was quantified from a minimum of three coverslips in each group. In all cases, the presented data are expressed as an average+/−the standard deviation.

### ii) Characterization of HNP1 Progenitor Cells

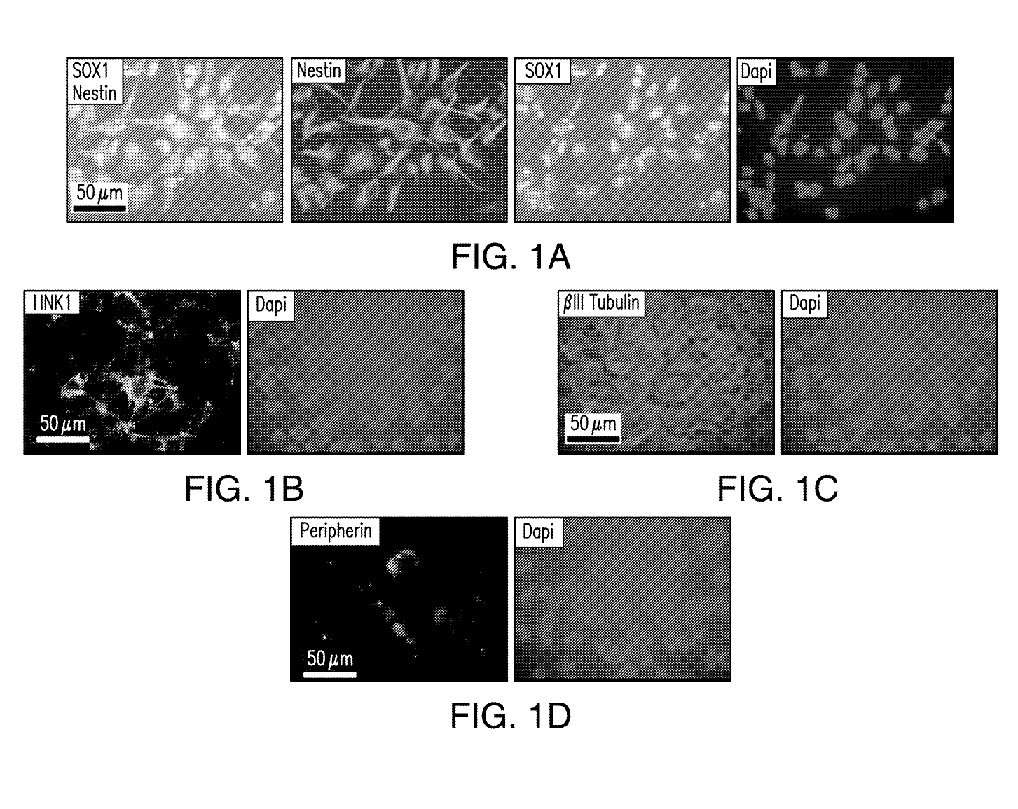

hNP1 cells were derived from the human embryonic stem cell line WA09 and were cultured as adherent monolayers. To establish a baseline composition before the induction of differentiation, the undifferentiated hNP1 cells were examined using established markers for neural progenitor cells, neural crest cells, and sensory neurons. The hNP1 cells were plated on DETA-coated glass coverslips at 400 cells/mm² in growth medium and were fixed for immunostaining two (2) days later. FIG. 1 shows that all the cells were positive for the neural progenitor markers nestin and SOX1 (FIG. 1A). Conversely, the cells were negative for both the neural crest cell marker HNK1 (FIG. 1B) and the general neuronal marker βIII tubulin (FIG. 1C). The cells were also negative for the sensory neuron marker peripherin (FIG. 1D). The results from the immunostaining confirmed that the hNP1 cell line provided a pure population of neural progenitors.

iii) Induction of Sensory Neurons from HNP1 Cells

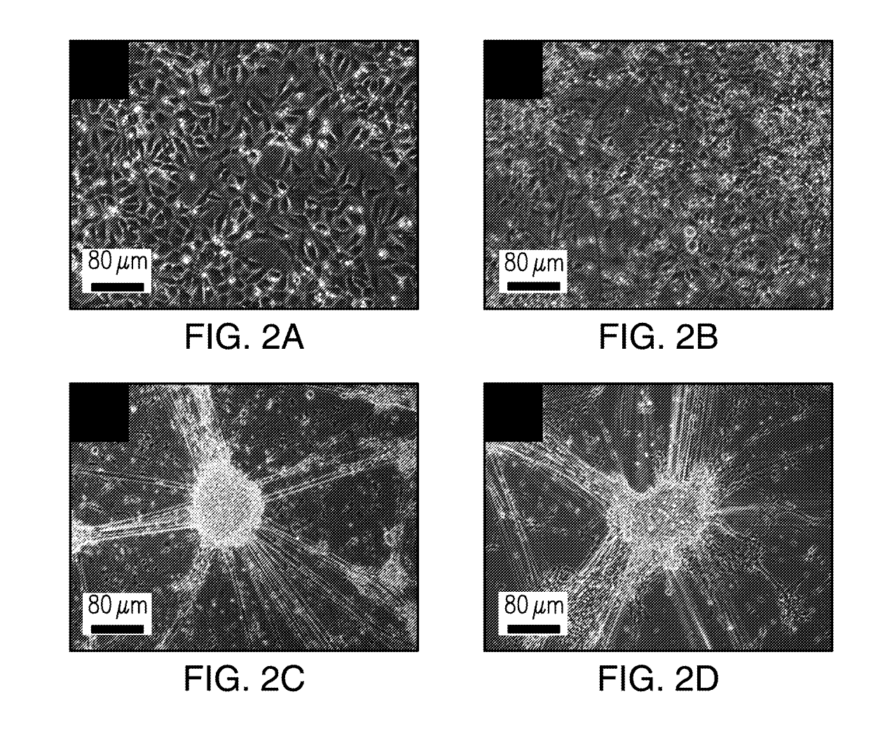

The hNP1 cell line was induced to generate sensory neurons by treating the cells with trophic factors and/or chemicals that had previously been shown to be important for the generation of sensory neurons (i.e., Noggin, SB431542, and Wnt1). (Hari et al., 2002; Menendez et al., 2011; Lee et al., 2010; Lee et al., 2004). From day 8 after differentiation, most of the cells had gradually adopted a neural morphology with smaller somas and long processes (FIG. 2B). From day 12 after differentiation, the medium was switched to a hSN differentiation medium (Table 1) and as differentiation continued, the cells with neuronal morphology became more prominent. These neurons tended to form clusters with their axons aligning uniformly and were located between the clustered cell bodies (FIG. 2C). This feature was similarly observed in cultures derived from rat embryonic DRGs in which sensory neurons formed clusters with their axons developing “highway” bundles traveling between individual clusters (FIG. 2D) (Guo et al., 2012).

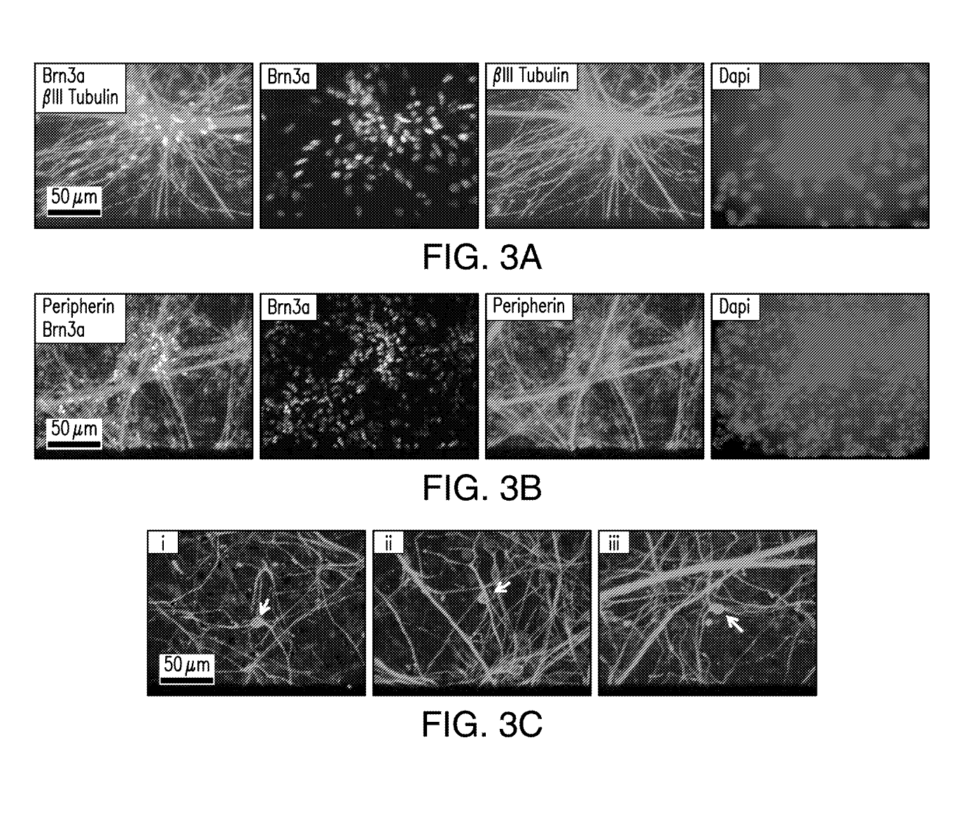

To confirm the induction of differentiation from the hNP1 progenitor cells to sensory neurons, the cultures were fixed and immunostained with a marker for sensory neurons (i.e., Brn3a) (Greenwood et al., 1999), a marker for peripheral neurons (i.e., peripherin), and a general neuronal marker (i.e., βIII tubulin). The immunocytochemical analysis indicated that there was a significant number of sensory neurons induced in these cultures. (FIG. 3A and FIG. 3B). Closer observation of the immunostained cells also revealed sensory neurons at different developmental stages: (i) bipolar neurons that were in an early stage of differentiation, (ii) cells with two axons moving closer together, indicating an intermediate stage, and (iii) pseudo-unipolar neurons, indicating a more mature stage. (FIG. 3C).

### iv) Analysis of Differentiation Products

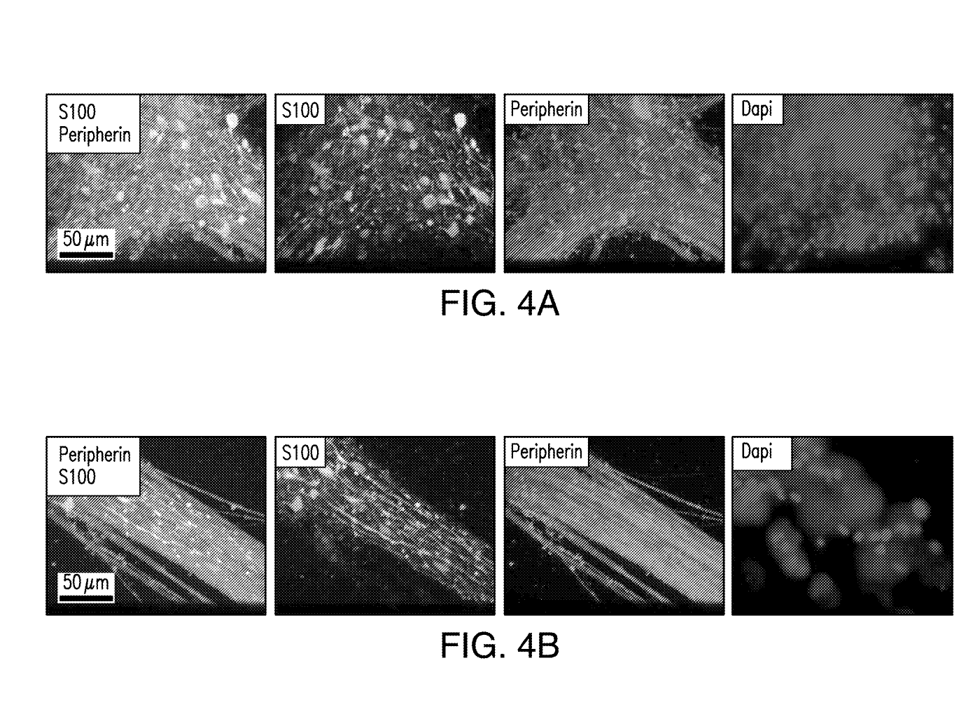

Following differentiation, the resulting cell cultures were subjected to additional characterization with additional cellular markers. First, as Schwann cells are also derived from neural crest stem cells and are closely related to sensory neurons in physical distribution and function, whether Schwann cells were generated was investigated Immunocytochemical analysis utilizing the Schwann cell marker S100 demonstrated the presence of an abundant number of Schwann cells in the differentiated culture. These Schwann cells were found in both the neuronal clusters (FIG. 4A) and in the axonal bundles (FIG. 4B). Quantification of the immunostaining indicated that, following differentiation, neurons that positively stained for βIII tubulin and peripherin accounted for 51.3+/−3.7% of the total number of cells and Schwann cells that positively stained for S100 accounted for 48.0+/−2.5% of the total number of cells in culture

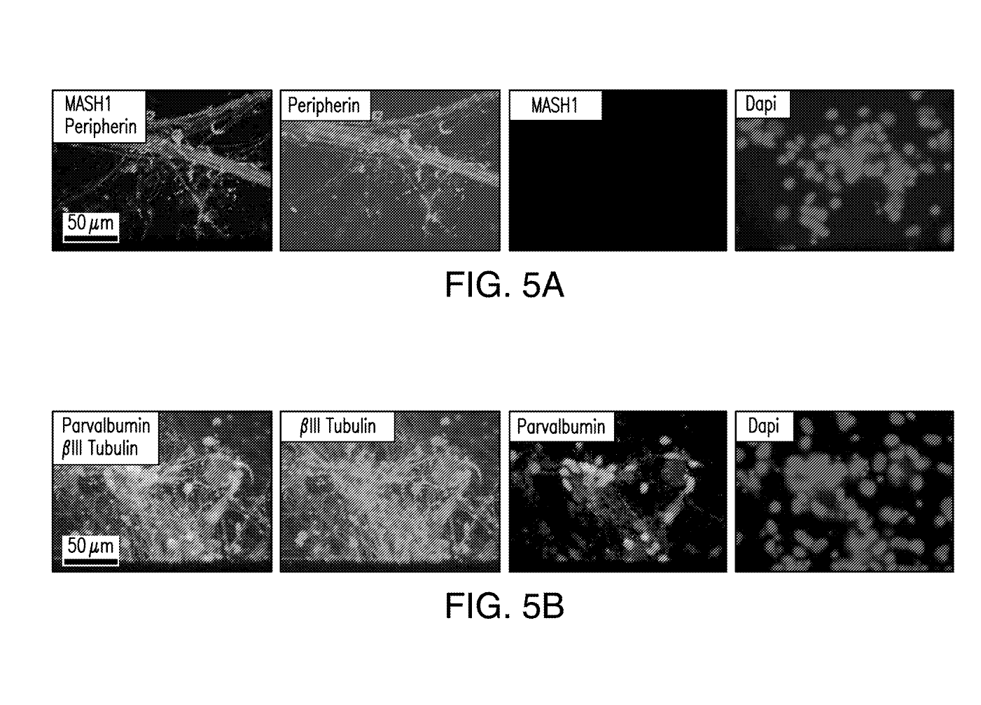

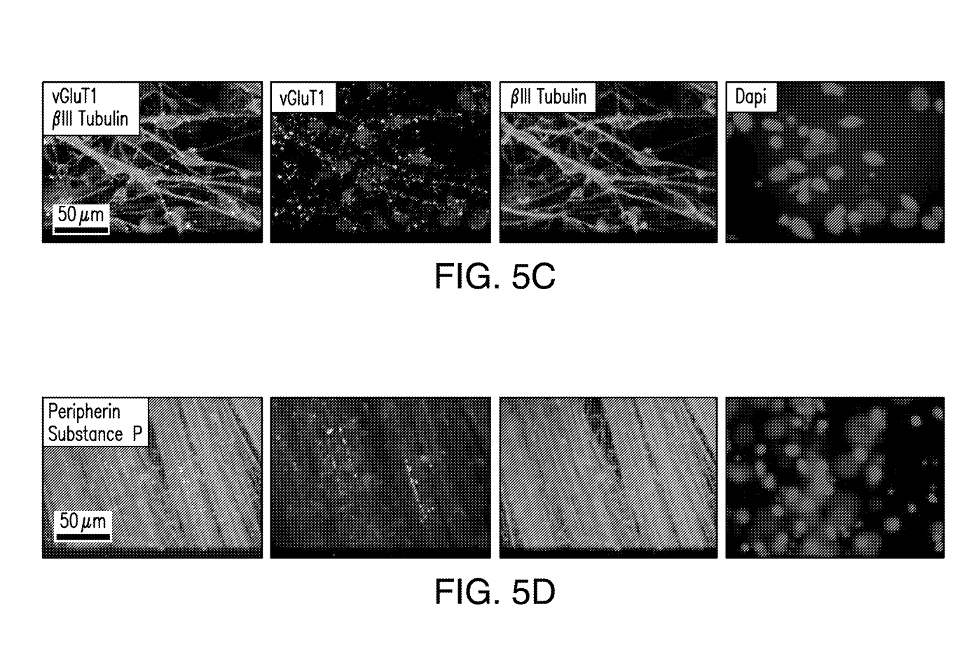

Since peripherin-positive neurons can be sensory or autonomic neurons, the number of autonomic neurons in the derived neuronal population was determined. The cultures were immunostained for MASH1, a transcription factor specific for autonomic neurons (Anderson et al., 1997), and were co-immunostained for peripherin. None of the peripherin-positive cells were MASH1-positive. However, all the examined peripherin-positive cells were Brn3a-positive. (FIG. 3B). These results indicated that all of the peripherin-positive neurons were sensory neurons. (FIG. 5A). To identify the type of the sensory neurons present in the culture, the differentiated cultures were further immunostained for parvalbumin, which is a marker for large type I proprioceptive sensory neurons. (Liu et al., 2008; Carr et al., 1989; Ichikawa et al., 2004). (FIG. 5A). Most of the neurons stained positively for parvalbumin, indicating that the neurons were sensory neurons in the type I category (FIG. 5B).

The cultures were further analyzed by immunocytochemistry for the expression of vGluT1. Glutamate is used as the neurotransmitter in the afferents of all DRG neurons, including both glutamatergic and peptidergic neurons. (Julius et al., 2001). Glutamate transporters (vGluT) are proteins located on synaptic vesicles that are responsible for packaging the neurotransmitter glutamate into vesicles. vGluT1 is mostly present in medium to large-sized, CGRP (calcitonin gene related peptide)-negative DRG neurons, which are type I sensory neurons. (Brumovsky et al., 2007). The vGluT1 positive result (FIG. 5C) also indicated that the differentiated neurons were primarily type I sensory neurons.

To determine whether any nociceptive sensory neurons were generated, the differentiated cultures were immunostained for substance P, which is the peptidergic neurotransmitter for nociceptive sensory neurons. Very few substance P-positive fibers or neurons were identified in the differentiated cultures (<1%). (FIG. 5D). These data indicate that the differentiated neurons were mostly propioceptive sensory neurons.

### v) Electrophysiological Analysis of the Differentiated Sensory Neurons

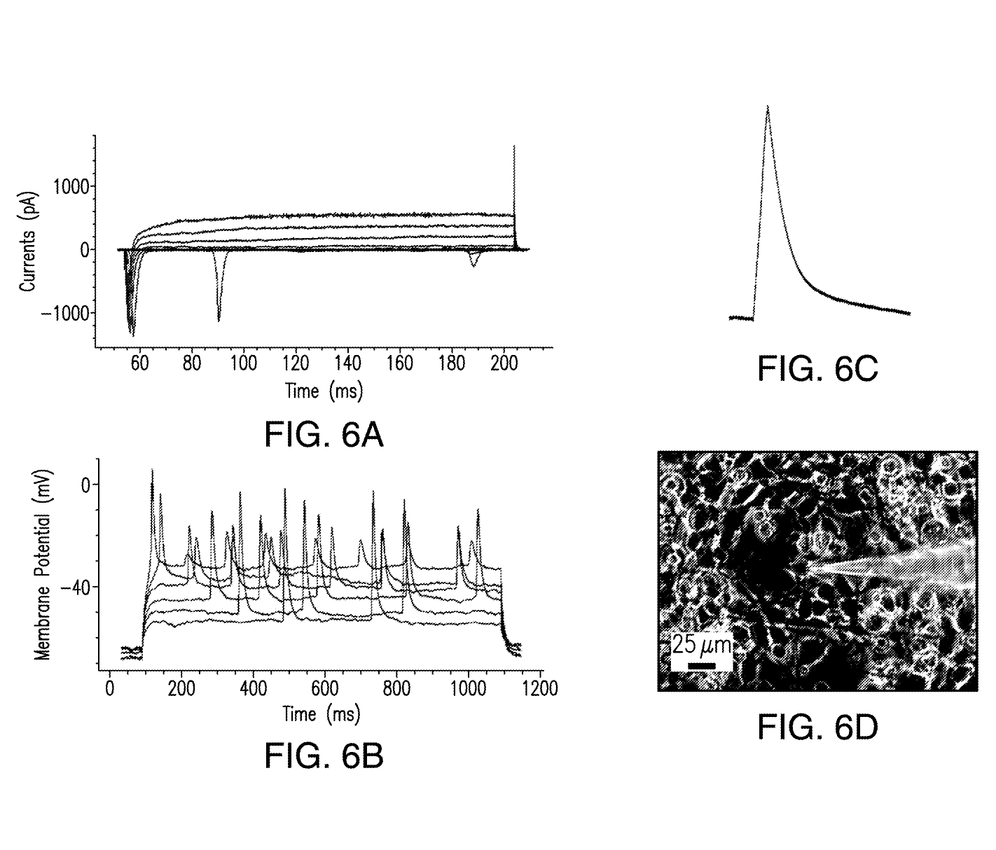

The functional maturation of the differentiated sensory neurons was determined by electrophysiological analysis. (FIG. 6). The voltage clamp analysis revealed that voltage-dependent currents were invoked. (FIG. 6A). Furthermore, the delay and dynamics of the inward and outward currents were consistent with sodium and delayed rectifier potassium currents. The maximum inward and outward currents reached values of −1150 pA and 728 pA, respectively. Prolonged stepped, current clamp recordings (0-100 pA×1 second) indicated that 23 of the 24 recorded cells were capable of firing action potentials (APs). Of the firing cells, 19 cells fired single APs and 4 cells displayed repetitive firing behavior. The maximum firing frequency among these cells was 9 Hz. (FIG. 6B). Single APs elicited by a brief saturated depolarization current had an amplitude of 95.4+/−22.4 mV. (FIG. 6C). Here, the reliable elicitation of APs and the presence of repetitive APs indicated that these sensory neurons were functional. The positive staining for vGluT1 indicated that the sensory neurons synthesized the correct neurotransmitter, which is an indicator of functional maturation.

vi) Derivation of Neural Crest Stem Cells from HNP1 Stem Cells

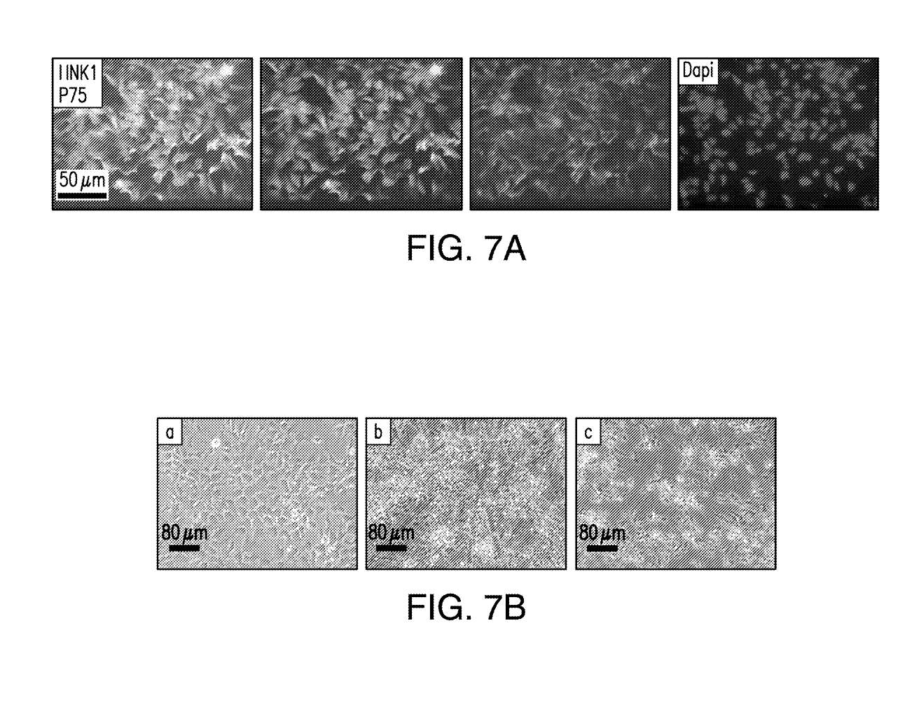

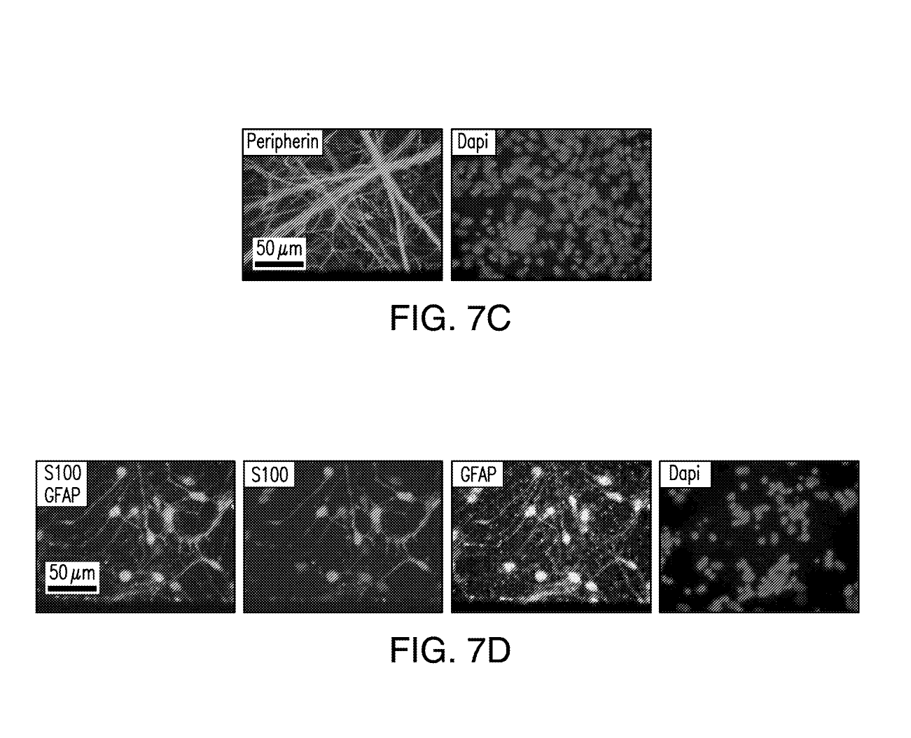

To determine whether NCSCs were generated during the sensory neuron induction protocol, the differentiated cells (day 10) were re-plated and were subjected to proliferation by including bFGF and EGF in the N2B medium (Lee et al., 2010). Treatment with bFGF was found to enhance the proliferation of NCSCs (Murphy et al., 1994) and the combination of bFGF with EGF promoted the neuronal fate induction from NCSCs (Garcez et al., 2009). After 2 passages, nearly all of the cultured cells presented a stem cell morphology and were active in proliferation. The immunostaining of the neural crest stem cells indicated that most cells were positive for HNK1 (90%) and P75 (99%). (FIG. 7A).

To determine whether these NCSCs retained the potential to generate sensory neurons, an aliquot of passage 4 cells were induced with either (i) a sensory neuron differentiation medium or (ii) a Schwann cell differentiation medium. After approximately 2 weeks, the cells under each differentiation condition demonstrated a different morphology and both were distinguishable from undifferentiated NCSCs. (FIG. 7B). An immunocytochemical analysis demonstrated that those NCSCs subjected to the sensory neuron differentiation protocol stained positively for the peripheral neuronal marker peripherin, which indicated the presence of a significant number of sensory neurons. (FIG. 7C). Similarly, an immunocytochemical analysis demonstrated that those NCSCs subjected to the Schwann cell differentiation protocol stained positively for the Schwann cell markers S100 and GFAP, which indicated the presence of a significant number of Schwann cells. (FIG. 7D). Therefore, these NCSCs retained the ability to generate sensory neurons and Schwann cells, both of which are integral components of dorsal root ganglion structures.

vii) Experimental Advantages

This data provided herein confirm that human neural progenitor cell line, hNP1, which was originally derived from the embryonic stem cell line WA09 (H9), can differentiate into functional sensory neurons and into neural crest stem cells (NCSCs). First, the presence of sensory neurons was confirmed by immunocytochemistry and the functional maturation of these sensory neurons was analyzed using electrophysiology. Second, as confirmed by the appropriate immunocytochemistry, the NCSCs preserved the ability to generate sensory neurons and Schwann cells. As described herein, the generation of functional sensory neurons as well as NCSCs provide valuable tools for the modeling and treatment of sensory neuron-related disorders.

The compositions and methods disclosed herein provide an efficient method for the generation of sensory neurons, Schwann cells, and NCSCs. Specifically, the generation of these cells was accomplished in an in vitro, adherent culture system. The culture system did not require multicellular aggregates or stromal cells. Furthermore, the culturing and the differentiation methods provided here better than other methods as the present methods eliminate contamination generated by feeder cells. In fact, the present methods eliminate the need for feeder cells. As disclosed herein, the induction of sensory neurons from the progenitor cells was faster than methods that use ESCs.

In the experiments disclosed herein, the methods directed ˜50% of the cultured cells to a sensory neuron fate and the other 50% of cultured cells to a Schwann cell fate. Conversely, only <1% and ˜10% of cultured cells developed into sensory neurons when the methods utilized ESCs (Pomp et al., 2005) or neurospheres (Brokhman et al., 2008). The data generated herein indicate that the disclosed methods represent a very efficient protocol. The NCSCs derived from this protocol demonstrated ˜90% purity without any subsequent sorting procedures, which is also an advantage over other systems (Jiang et al., 2009; Lee et al., 2008). Thus, this experiments and methods described herein represent several significant advancements for in the generation of sensory neurons and NC cells from undifferentiated cell sources.

It will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only, with a true scope and spirit of the invention being indicated by the following claims.

All of the compositions and/or methods disclosed and claimed herein can be made and executed without undue experimentation in light of the present disclosure. It will be apparent to those skilled in the art that various modifications and variations can be made in the present invention without departing from the scope or spirit of the invention.

### E. REFERENCES

Anderson D J, et al. Cell lineage determination and the control of neuronal identity in the neural crest. Cold Spring Harb Symp Quant Biol 1997; 62:493-504.
Brokhman I, et al. Peripheral sensory neurons differentiate from neural precursors derived from human embryonic stem cells. Differentiation 2008; 76:145-155.
Brumovsky P, et al. Expression of the vesicular glutamate transporters-1 and -2 in adult mouse dorsal root ganglia and spinal cord and their regulation by nerve injury. Neuroscience 2007; 147:469-490.
Carr P A, et al. Parvalbumin is highly colocalized with calbindin d28k and rarely with calcitonin gene-related peptide in dorsal root ganglia neurons of rat. Brain Res 1989; 497:163-170.
Chambers S M, et al. Highly efficient neural conversion of human es and ips cells by dual inhibition of smad signaling. Nat Biotech 2009; 27:275-280.
Das M, et al. Adult rat spinal cord culture on an organosilane surface in a novel serum-free medium. In Vitro Cell Dev Biol Anim 2005; 41:343-348.
Das M, et al. Electrophysiological and morphological characterization of rat embryonic motoneurons in a defined system. Biotechnol Prog 2003; 19:1756-1761.
Das M, et al. Embryonic motor neuron-skeletal muscle co-culture in a defined system. Neuroscience 2007; 146:481-488
Fernandez-Valle C, et al. Schwann cells degrade myelin and proliferate in the absence of macrophages: Evidence from in vitro studies of wallerian degeneration. J Neurocytol 1995; 24:667-679.
Gao B X, et al. Development of ionic currents underlying changes in action potential waveforms in rat spinal motoneurons. J Neurophysiol 1998; 80:3047-3061.
Garcez R C, et al. Epidermal growth factor (ego promotes the in vitro differentiation of neural crest cells to neurons and melanocytes. Cell Mol Neurobiol 2009; 29:1087-1091.
Gerrard L, et al. Differentiation of human embryonic stem cells to neural lineages in adherent culture by blocking bone morphogenetic protein signaling. Stem Cells 2005; 23:1234-1241.
Greenwood A L, et al. Identification of dividing, determined sensory neuron precursors in the mammalian neural crest. Development 1999; 126:3545-3559.
Groves M J, et al. Chapter 31—pathology of peripheral neuron cell bodies. In: Peter J D, Thomas P K, editors. Peripheral neuropathy (fourth edition). Philadelphia: W.B. Saunders, 2005. p. 683-732.
Guo X, et al. Tissue engineering the monosynaptic circuit of the stretch reflex arc with co-culture of embryonic motoneurons and proprioceptive sensory neurons. Biomaterials 2012; 33:5723-5731.
Guo X F, et al. Characterization of a human fetal spinal cord stem cell line NSI-566RSC and its induction to functional motoneurons. J Tissue Eng Regen Med 2010; 4:181-193.
Hari L, et al. Lineage-specific requirements of beta-catenin in neural crest development. J Cell Biol 2002; 159:867-880.
Hickman J J, et al. Rational Pattern Design for in-Vitro Cellular Networks Using Surface Photochemistry. J Vac Sci Technol A-Vac Surf Films 1994; 12:607-616.
Hjerling-Leffler J, et al. The boundary cap: A source of neural crest stem cells that generate multiple sensory neuron subtypes. Development 2005; 132:2623-2632.
Ichikawa H, et al. Effect of brn-3a deficiency on parvalbumin-immunoreactive primary sensory neurons in the dorsal root ganglion. Brain Res Dev Brain Res 2004; 150:41-45.
Jiang X H, et al. Isolation and characterization of neural crest stem cells derived from in vitro-differentiated human embryonic stem cells. Stem Cells Dev 2009; 18:1059-1070.
Julius D, et al. Molecular mechanisms of nociception. Nature 2001; 413:203-210.
Kim J Dopamine neurons derived from embryonic stem cells function in an animal model of Parkinson's disease. Nature 2002; 418:50-56.
Kim S U, et al. Production of Immortalized Human Neural Crest Stem Cells. Neural stem cells: Methods and Protocols: Humana Press, 2002. p. 55-64.
Lamb T M, et al. Neural induction by the secreted polypeptide noggin. Science 1993; 262:713-718.
Le Douarin N M, et al. Multipotentiality of the neural crest. Curr Opin Genet Dev 2003; 13:529-536.
Lee A. Isolation of neural stem cells from the postnatal cerebellum. Nat Neurosci 2005; 8:723-729.
Lee G, et al. Derivation of neural crest cells from human pluripotent stem cells. Nat Protoc 2010; 5:688-701.
Lee G, et al. Isolation and directed differentiation of neural crest stem cells derived from human embryonic stem cells. Nat Biotechnol 2007; 25:1468-1475.
Lee H-Y, et al. Instructive role of wnt/beta-catenin in sensory fate specification in neural crest stem cells. Science 2004; 303:1020-1023.
Li X J, et al. Specification of motoneurons from human embryonic stem cells. Nat Biotechnol 2005; 23:215-221.
Lisak R P, et al. The role of cytokines in schwann cell damage, protection, and repair. J Infect Dis 1997; 176:S173-S179.
Liu J, et al. Electrophysiological and immunocytochemical characterization of DRG neurons on an organosilane surface in serum free medium. In Vitro Cell Dev Biol 2008; 44:162-168.
McMahon J A, et al. Noggin-mediated antagonism of BMP signaling is required for growth and patterning of the neural tube and somite. Genes Dev 1998; 12:1438-1452.
Menendez L, et al. Wnt signaling and a smad pathway blockade direct the differentiation of human pluripotent stem cells to multipotent neural crest cells. Proc Natl Acad Sci USA 2011; 108:19240-19245.
Mizuseki K, et al. Generation of neural crest-derived peripheral neurons and floor plate cells from mouse and primate embryonic stem cells. Proc Natl Acad Sci USA 2003; 100:5828-5833.
Murphy M, et al. Fgf2 regulates proliferation of neural crest cells, with subsequent neuronal differentiation regulated by lif or related factors. Development 1994; 120:3519-3528.
Nat R. Cortical network from human embryonic stem cells. J Cell Mol Med 2011; 15:1429-1431.
Perrier A L, et al. Derivation of midbrain dopamine neurons from human embryonic stem cells. Proc Natl Acad Sci USA 2004; 101:12543-12548.
Pomp O, et al. Generation of peripheral sensory and sympathetic neurons and neural crest cells from human embryonic stem cells. Stem Cells 2005; 23:923-930.
Pomp O, et al. PA6-induced human embryonic stem cell-derived neurospheres: a new source of human peripheral sensory neurons and neural crest cells. Brain Res 2008; 1230:50-60.
Schulz T C, et al. Differentiation of human embryonic stem cells to dopaminergic neurons in serum-free suspension culture. Stem Cells 2004; 22:1218-1238.
Sghirlanzoni A, et al. Sensory neuron diseases. Lancet Neurol 2005; 4:349-361.
Shah N M, et al. Alternative neural crest cell fates are instructively promoted by tgf-beta superfamily members. Cell 1996; 85:331-343.
Sheikh S I, et al. The dorsal root ganglion under attack: The acquired sensory ganglionopathies. Pract Neurol 2010; 10:326-334.
Singh R P, et al. Retentive multipotency of adult dorsal root ganglia stem cells. Cell Transplant 2009; 18:55-68.
Smith J R, et al Inhibition of activin/nodal signaling promotes specification of human embryonic stem cells into neuroectoderm. Dev Biol 2008; 313:107-117.

What is claimed is:

1. A method of generating sensory cells or neural crest stem cells, comprising:
(i) proliferating a population of neural progenitor cells;
(ii) dissociating the population of proliferated neural progenitor cells;
(iii) replating the population of dissociated neural progenitor cells;
(iv) expanding the population of replated neural progenitor cells; and
(v) initiating differentiation of the population of expanded neural progenitor cells into sensory neurons or neural crest stem cells.

2. The method of claim 1, further comprising
(vi) promoting differentiation of the population of differentiating neural progenitor cells into sensory neurons.

3. The method of claim 1, further comprising
(vi) dissociating the population of differentiating neural progenitor cells; and
(vii) replating the population of dissociated and differentiating neural progenitor cells,
(vi) promoting differentiation of the population of differentiating neural progenitor cells into neural crest stem cells.

4. The method of claim 3, wherein the cells are replated on glass coverslips pre-coated with poly-ornathine/laminin/fibronectin at a density of 200 cells/mm².

5. The method of claim 4, wherein the replated cells are maintained in N2B medium comprising bFGF and hEGF.

6. The method of claim 4, wherein the N2B medium comprising bFGF and hEGR is changed every two days.

7. The method of claim 1, wherein the neural progenitor cells are human neural progenitor cells.

8. The method of claim 1, wherein the neural progenitor cells are non-human neural progenitor cells.

9. The method of claim 1, wherein the neural progenitor cells are hNP1 cells.

10. The method of claim 1, wherein feeder cells are not used.

11. The method of claim 1, wherein proliferating the population of neural progenitor cells comprises maintaining the cells in proliferation medium comprising bFGF.

12. The method of claim 11, wherein the proliferation medium is changed every other day.

13. The method of claim 1, wherein proliferating the population of neural progenitor cells comprises 3-5 days.

14. The method of claim 1, wherein proliferating the population of neural progenitor cells continues until the cells reach 100% confluence.

15. The method of claim 1, wherein dissociating the population of neural progenitor cells comprises manual dissociation, chemical dissociation, or a combination thereof.

16. The method of claim 3, wherein dissociating the population of neural progenitor cells comprises manual dissociation, chemical dissociation, or a combination thereof.

17. The method of claim 1, wherein replating the population of neural progenitor cells comprises replating the cells onto glass coverslips at a density of 400 cells/mm².

18. The method of claim 16, wherein the glass coverslips are pre-coated with DETA, or poly-ornathine/laminin/fibronectin, or a combination thereof.

19. The method of claim 1, wherein expanding the population of neural progenitor cells comprises maintaining the population of cells in proliferation medium.

20. The method of claim 19, wherein the cells are maintained in proliferation medium for 2-3 days.

21. The method of claim 1, wherein expanding the population of neural progenitor cells continues until the cells reach approximately 90% confluence.

22. The method of claim 1, wherein initiating differentiation of the population of neural progenitor cells comprises:
a. replacing proliferation medium with KSR medium;
b. replacing KSR medium with N2B medium; and
c. replacing N2B medium with a differentiation medium.

23. The method of claim 22, wherein KSR medium comprises knockout DMEM, KSR, SB435142, Noggin, L-glutamine, MEM, penicillin/streptomycin, and beta-mercaptoethanol.

24. The method of claim 22, wherein replacing KSR medium with N2B medium occurs gradually.

25. The method of claim 24, wherein replacing KSR medium with N2B medium comprises at least 10 days.

26. The method of claim 25, wherein on Day 0, the medium comprises 100% KSR medium.

27. The method of claim 25, wherein on Day 1, the medium comprises 100% KSR medium.

28. The method of claim 25, wherein on Day 2, the medium comprises 75% KSR medium and 25% N2B.

29. The method of claim 25, wherein on Day 3, the medium comprises 75% KSR medium and 25% N2B.

30. The method of claim 25, wherein on Day 4, the medium comprises 50% KSR medium and 50% N2B.

31. The method of claim 25, wherein on Day 5, the medium comprises 50% KSR medium and 50% N2B.

32. The method of claim 25, wherein on Day 6, the medium comprises 25% KSR medium and 75% N2B.

33. The method of claim 25, wherein on Day 7, the medium comprises 25% KSR medium and 75% N2B.

34. The method of claim 25, wherein on Day 8, the medium comprises 100% N2B.

35. The method of claim 25, wherein on Day 9, the medium comprises 100% N2B.

36. The method of claim 25, wherein on Day 10, the medium comprises and 100% N2B.

37. The method of claim 25, wherein the concentration of SB435142 and Noggin remain constant.

38. The method of claim 22, wherein differentiation medium comprises N2B medium comprising BDNF, L-ascorbic acid, GDNF, NGF, NT-3, cAMP, and Wnt-1.

39. The method of claim 22, wherein one-third of the differentiation medium is changed every two days.

40. The method of claim 22, wherein the cells are maintained in the differentiation medium for at least 2 days.

41. Use of a sensory neuron made using the method of any one of claims 1-2 or claims 7-40.

42. A sensory neuron made using the method of any one of claims 1-2 or claims 7-40.

43. Use of a neural crest stem cell made by the method of any one of claim 1 or claims 3-37.

44. A neural crest stem cell made by the method of any one of claim 1 or claims 3-37.

45. A method of generating sensory neurons, comprising:
(i) proliferating a population of neural crest stem cells; and
(ii) initiating differentiation of the population of neural crest stem cells into sensory neurons.

46. The method of claim 45, wherein feeder cells are not used.

47. The method of claim 45, wherein proliferating the population of neural crest stem cells comprises maintaining the cells in N2B medium comprising bFGF and hEGF.

48. The method of claim 47, wherein N2B medium is changed every other day.

49. The method of claim 47, wherein maintaining the cells in N2B medium continues until the cells reach about 100% confluence.

50. The method of claim 45, wherein initiating differentiation of the population of neural crest stem cells comprises replacing N2B medium with a differentiation medium.

51. The method of claim 50, wherein differentiation medium comprises N2B medium comprising BDNF, L-ascorbic acid, GDNF, NGF, NT-3, cAMP, and Wnt-1.

52. The method of claim 50, wherein one-third of the medium is changed every two days.

53. The method of claim 45, wherein the population of neural crest stem cells are generated according to the method of any one of claim 1 or claims 3-37.

54. Use of a sensory neuron made by the method of any one of claims 45-53.

55. A sensory neuron made using the method of any one of claims 45-53.

56. A method of generating Schwann cells, the method comprising:
(i) proliferating a population of neural crest stem cells; and
(ii) initiating the differentiation of the population of neural crest stem cells into Schwann cells.

57. The method of claim 56, wherein feeder cells are not used.

58. The method of 56, wherein proliferating the population of neural crest stem cells comprises maintaining the cells in N2B medium comprising bFGF and hEGF.

59. The method of claim 58, wherein N2B medium is changed every two days.

60. The method of claim 58, wherein maintaining the cells in N2B medium continues until the cells reach about 100% confluence.

61. The method of claim 56, wherein initiating the differentiation of the population of neural crest stem cells comprises replacing the N2B medium with differentiation medium.

62. The method of claim 61, wherein differentiation medium comprises N2B medium comprising CNTF, neuregulin, bFGF, and cAMP.

63. The method of claim 61, wherein one-third of the medium is changed every two days.

64. The method of claim 56, wherein the population of neural crest stem cells are generated according to the method of any one of claim 1 or claims 3-37.

65. Use of a Schwann cell generated by the method of any one of claims 56-64.

66. A Schwann cell made by the method of any one of claims 56-64.

67. A kit, comprising:
(i) a population of neural progenitor cells; and
(ii) instructions for differentiating the population of neural progenitor cells into sensory neurons.

68. The kit of claim 67, wherein the neural progenitor cells are human neural progenitor cells.

69. The kit of claim 67, wherein the neural progenitor cells are non-human neural progenitor cells.

70. The kit of claim 67, wherein the neural progenitor cells are hNP1 cells.

71. The kit of claim 67, further comprising one or more mediums.

72. The kit of claim 71, wherein the one or more mediums comprise:
a first medium, wherein the first medium is used during proliferation of the population of neural progenitor cells and wherein the first medium is used during expansion of the population of neural progenitor cells;
a second medium, wherein the second medium is used during induction of differentiation of the population of neural progenitor cells;
a third medium, wherein the third medium is used during promotion of differentiation of the population of neural progenitor cells; and
a fourth medium, wherein the fourth medium is used during promotion of differentiation of the population of neural progenitor cells.

73. The kit of claim 72, wherein the first medium comprises AB2 basal medium comprising bFGF.

74. The kit of claim 72, wherein the second medium comprises knockout DMEM, KSR, L-glutamine, MEM, penicillin/streptomycin, beta-mercaptoethanol, SB435142, and noggin.

75. The kit of claim 72, wherein the third medium comprises N2B medium.

76. The kit of claim 72, wherein the fourth medium comprises N2B medium comprising BDNF, L-ascorbic acid, GDNF, NGF, NT-3, cAMP, and Wnt-1.

77. The kit of claim 67, further comprising glass coverslips, wherein the glass coverslips are pre-coated with DETA, poly-ornathine/laminin/fibronectin, or a combination thereof.

78. A kit, comprising:
(i) a population of neural progenitor cells; and
(ii) instructions for differentiating the population of neural progenitor cells into neural crest stem cells.

79. The kit of claim 78, wherein the neural progenitor cells are human neural progenitor cells.

80. The kit of claim 78, wherein the neural progenitor cells are non-human neural progenitor cells.

81. The kit of claim 78, wherein the neural progenitor cells are hNP1 cells.

82. The kit of claim 78, further comprising one or more mediums.

83. The kit of claim 83, wherein the one or more mediums comprise:
a first medium, wherein the first medium is used during proliferation of the population of neural progenitor cells and wherein the first medium is used during expansion of the population of neural progenitor cells;
a second medium, wherein the second medium is used during induction of differentiation of the population of neural progenitor cells;
a third medium, wherein the third medium is used during promotion of differentiation of the population of neural progenitor cells; and
a fourth medium, wherein the fourth medium is used during promotion of differentiation of the population of neural progenitor cells.

84. The kit of claim 83, wherein the first medium comprises AB2 basal medium comprising bFGF.

85. The kit of claim 83, wherein the second medium comprises knockout DMEM, KSR, L-glutamine, MEM, penicillin/streptomycin, beta-mercaptoethanol, SB435142, and noggin.

86. The kit of claim 83, wherein the third medium comprises N2B medium.

87. The kit of claim 83, wherein the fourth medium comprises N2B medium comprising bFGF and hEGF.

88. The kit of claim 78, further comprising glass coverslips, wherein the glass coverslips are pre-coated with DETA, or poly-ornathine/laminin/fibronectin, or a combination thereof.

89. A kit, comprising:
(i) a population of neural crest stem cells; and
(ii) instructions for differentiating the population of neural crest stem cells into sensory neuron, Schwann cells, or both.

90. The kit of claim 89, wherein the population of neural crest stem cells are generated according to the method of any one of claim 1 or claims 3-37.

91. The kit of claim 89, further comprising one or more mediums.

92. The kit of claim 91, wherein the one or more mediums comprise:
a first medium, wherein the first medium is used during proliferation of the population of neural crest stems cells;
a second medium, wherein the second medium is used during differentiation of the population of neural crest stems cells into sensory neurons; and
a third medium, wherein the third medium is used during differentiation of the population of neural crest stem cells into Schwann cells.

93. The kit of claim 92, wherein the first medium comprises N2B medium comprising bFGF and hEGF.

94. The kit of claim 92, wherein the second medium comprises N2B medium comprising BDNF, L-ascorbic acid, GDNF, NGF, NT-3, cAMP, and Wnt-1.

95. The kit of claim 92, wherein the third medium comprises N2B medium comprising CNTF, neuregulin, bFGF, and cAMP.

96. The kit of claim 89, further comprising glass coverslips, wherein the glass coverslips are pre-coated with DETA, or poly-ornathine/laminin/fibronectin, or a combination thereof.
